(19) United States
(12) Patent Application Publication
(10) Pub. No.: US 2006/0194193 A1
(43) Pub. Date: Aug. 31, 2006

(54) Biological sample culturing and observation system, incubator, supplying device, and culture vessel

(75) Inventors: Hiroshi Tsuruta, Tokyo (JP); Kayuri Muraki, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, TOKYO (JP)

(21) Appl. No.: 11/287,864

(22) Filed: Nov. 28, 2005

(30) Foreign Application Priority Data
Nov. 29, 2004 (JP) 2004-344568
Nov. 18, 2005 (JP) 2005-334726

(51) Int. Cl. C12Q 1/00; C12M 1/34

(52) U.S. Cl. 435/4; 435/287.1

(57) ABSTRACT

The present invention provides a biological sample culturing and observation system which includes: an incubator in which a biological samples are housed, and whose interior is maintained in a culturing environment satisfying predetermined conditions, and the interior is isolated from the outside; an observation optical system that from outside the incubator optically observes the biological sample via the incubator; a light blocking device that blocks external light that is irradiated on the biological sample and within the visual field of a observation optical system; and a supply device that selectively supplies a liquid or gas to the biological sample inside the incubator from a plurality of holding vessels that individually hold a plurality of different types of liquid or gas that are necessary for the culturing.

### BACKGROUND OF THE INVENTION

1. Field of the Invention

The present invention relates to a biological sample culturing and observation system that is used to detect information based on reactions of biological samples such as, for example, cultured cells, and to an incubator, a supply device, and a culture vessel that are used in this biological sample culturing and observation system.

Priority is claimed on Japanese Patent Application No. 2004-344568, filed Nov. 29, 2004, and Japanese Patent Application No. 334726, filed Nov. 18, 2005, the contents of which are incorporated herein by reference.

2. Description of Related Art

Recent years have seen advances in gene analysis technology, and gene arrangements in a number of living organisms, including humans, have been revealed. Moreover, the causal relationship between analyzed gene products (proteins) and illnesses is also becoming gradually clearer. In the future, in order to more comprehensively and statistically analyze various proteins and genes, the research and development of examination devices that use biological samples such as cells will show dramatic advances.

In such examination devices, because it is necessary to detect predetermined information while culturing biological samples such as cells for an extended period, for example, a biological sample culturing and observation system has been proposed in which, using an observation optical system such as a microscope, biological samples are observed while they are being cultured.

In a biological sample culturing and observation system in which cells are observed as they are being cultured over an extended period on the stage of an observation optical system, it is necessary to replace the culture solution during the observation in order that waste matter (lactic acid and the like) that is discharged into the culture solution from the cells does not exceed a fixed concentration.

However, if a culturing vessel is opened and the culture solution is replaced on a stage that is not kept uncontaminated, there is a possibility that the interior of the culturing vessel will become contaminated. Naturally, accurate information cannot be obtained if an observation is performed while the interior of the culturing vessel is in a contaminated state. Moreover, if a culture solution is replaced on a clean bench that has been provided in a different location from the stage, then although it is possible to avoid contamination of the interior of the culturing vessel, the position of the biological sample being observed before the replacement of the culture solution is different from the position thereof after the replacement of the culture solution. As a result, continuous observation is not possible. If, on the other hand, culturing is performed using a large enough volume of culturing solution that matter transmitted from the cells and the like does not exceed a predetermined concentration, then the proliferation potency of the cells is reduced.

Therefore, a biological sample culturing and observation system has been proposed (see, for example, Japanese Patent Application, First Publication No. 2004-113092) in which a cell culturing chip is placed under an observation optical system such as a microscope and, while new culture solution is being supplied via a supply port to a well inside the cell culturing chip, culture solution is discharged from the well via a discharge port. Accordingly, the well interior can be held in a constant environment. By maintaining this state, it is possible to observe cells inside the cell culturing chip.

### SUMMARY OF THE INVENTION

The present invention is a biological sample culturing and observation system in which temporal changes in a cultured biological sample are observed while the biological sample is being cultured, comprising: an incubator in which the biological samples are housed, and whose interior is maintained in a culturing environment satisfying predetermined conditions, and the interior is isolated from the outside; an observation optical system that from outside the incubator optically observes the biological sample via the incubator; a light blocking device that blocks external light that is irradiated on the biological sample and within the visual field of the observation optical system; and a supply device that selectively supplies a liquid or gas to the biological sample inside the incubator from a plurality of holding vessels that individually hold a plurality of different types of liquid or gas that are necessary for the culturing.

The present invention is an incubator that is used in a biological sample culturing and observation system in which temporal changes in a cultured biological sample are optically observed using an observation optical system while the biological sample is being cultured, wherein the biological sample culturing and observation system comprising: an observation optical system that optically observes the biological sample; a light blocking device that blocks external light that is irradiated on the biological sample and within the visual field of the observation optical system; and a supply device that selectively supplies a liquid or gas to the biological sample from a plurality of holding vessels that individually hold a plurality of different types of liquid or gas that are necessary for culturing the biological sample, and wherein the biological sample that is optically observed from the outside using the observation optical system is contained inside the incubator, and the biological sample is cultured by maintaining the interior of the incubator isolated from the outside in an environment fulfilling predetermined conditions.

The present invention is a supply device for liquid or gas that is used in a biological sample culturing and observation system in which temporal changes in a cultured biological sample are observed while the biological sample is being cultured, wherein the biological sample culturing and observation system comprising: an incubator whose interior is maintained in a culturing environment whose conditions have been predetermined and that is isolated from the outside, with the biological samples being housed in this interior; an observation optical system that from outside the incubator optically observes the biological sample via the incubator; and a light blocking device that blocks external light that is irradiated on the biological sample and is within the visual field of the observation optical system, and wherein the liquid or gas is selectively supplied to the biological sample contained inside the incubator from a plurality of holding vessels that individually hold a plurality of different types of liquid or gas that are necessary for the culturing.

The present invention is a culturing vessel that is contained inside the incubator of a biological sample culturing and observation system in which temporal changes in a cultured biological sample are observed while the biological sample is being cultured, wherein the biological sample culturing and observation system comprising: an incubator whose interior is maintained in a culturing environment whose conditions have been predetermined and that is isolated from the outside, with the biological samples being housed in this interior; an observation optical system that from outside the incubator optically observes the biological sample via the incubator; a light blocking device that blocks external light that is irradiated on the biological sample and within the visual field of the observation optical system; and a supply device that selectively supplies a liquid or gas to the biological sample contained inside the incubator from a plurality of holding vessels that individually hold a plurality of different types of liquid or gas that are necessary for culturing the biological sample, and wherein the culturing vessel comprises: a culturing section that holds the biological sample; a light transmitting section that transmits light that is necessary for the optical observation onto an optical path between the observation optical system and the biological sample that is held in the culturing section; a first flow path that supplies the liquid or the gas from the supply device to the biological sample that is held in the culturing section; and a second flow path that discharges the liquid or gas that was supplied to the biological sample to the outside.

### BRIEF DESCRIPTION OF THE DRAWINGS

FIG. 1 is a schematic structural view showing a first embodiment of the biological sample culturing and observation system according to the present invention.

FIG. 2 is a plan view of the incubator shown in FIG. 1 and shows a state in which a well plate is installed.

FIG. 3 is a plan view of the incubator shown in FIG. 1 and shows a state in which a dedicated culturing vessel is installed.

FIG. 4 is a side view of the incubator shown in FIG. 1.

FIGS. 5A and 5B are views showing an example of a flow path switching valve.

FIGS. 6A, 6B, and 6C are views showing another example of a flow path switching valve.

FIG. 7 is a view showing another example of a flow path switching valve.

FIGS. 8A and 8B are views showing another example of a flow path switching valve.

FIG. 9 is a view illustrating an assembly method for assembling a supply device.

FIG. 10 is a flowchart illustrating an assembly method for assembling a supply device.

FIG. 11 is a view illustrating an assembly method for assembling a supply device.

FIG. 12 is a cross-sectional view showing an example of a culturing vessel that is installed inside the incubator shown in FIG. 1.

FIG. 13 is a bottom view showing an example of a culturing vessel that is installed inside the incubator shown in FIG. 1.

FIG. 14 is a cross-sectional view showing another example of a culturing vessel that is installed inside the incubator shown in FIG. 1.

FIG. 15 is a cross-sectional view showing another example of a culturing vessel that is installed inside the incubator shown in FIG. 1.

FIG. 16 is a cross-sectional view showing another example of a culturing vessel that is installed inside the incubator shown in FIG. 1.

FIG. 17 is a cross-sectional view showing another example of a culturing vessel that is installed inside the incubator shown in FIG. 1.

FIG. 18 is a cross-sectional view showing another example of a culturing vessel that is installed inside the incubator shown in FIG. 1.

FIG. 19 is a cross-sectional view showing another example of a culturing vessel that is installed inside the incubator shown in FIG. 1.

FIG. 20 is a cross-sectional view showing another example of a culturing vessel that is installed inside the incubator shown in FIG. 1.

FIG. 21 is a cross-sectional view showing another example of a culturing vessel that is installed inside the incubator shown in FIG. 1.

FIG. 22 is a cross-sectional view showing another example of a culturing vessel that is installed inside the incubator shown in FIG. 1.

FIG. 23 is a cross-sectional view showing another example of a culturing vessel that is installed inside the incubator shown in FIG. 1.

FIG. 24 is a cross-sectional view showing another example of a culturing vessel that is installed inside the incubator shown in FIG. 1.

FIG. 25 is a schematic structural view of a cassette-type tank showing a variant example of the first embodiment of the biological culturing and observation system according to the present invention.

FIG. 26 is a cross-sectional view showing the structure of the switching valve used in FIG. 25.

FIG. 27 is a schematic structural view showing a variant example of the first embodiment of the biological sample culturing and observation system according to the present invention.

FIG. 28 is a schematic structural view showing a variant example of the first embodiment of the biological sample culturing and observation system according to the present invention.

FIG. 29 is a schematic structural view of a cassette-type tank showing a second embodiment of the biological culturing and observation system according to the present invention.

FIG. 30 is a plan view of the incubator shown in FIG. 29 and shows a state in which a well plate is installed.

FIG. 31 is a plan view of the incubator shown in FIG. 29 and shows a state in which a dedicated culturing vessel is installed.

FIG. 32 is a cross-sectional view showing an example of a culturing vessel that is installed inside the incubator shown in FIG. 29.

FIG. 33 is a cross-sectional view showing another example of a culturing vessel that is installed inside the incubator shown in FIG. 29.

FIG. 34 is a cross-sectional view showing another example of a culturing vessel that is installed inside the incubator shown in FIG. 29.

FIG. 35 is a cross-sectional view showing another example of a culturing vessel that is installed inside the incubator shown in FIG. 29.

FIG. 36 is a cross-sectional view showing an example of a cover for a well plate that is installed inside the incubator shown in FIG. 29.

FIG. 37 is a view as seen from the well plate side of the well plate cover shown in FIG. 36.

FIG. 38 is a cross-sectional view showing an example of a cover for a well plate that is installed inside the incubator shown in FIG. 29.

FIG. 39 is a view as seen from the well plate side of the well plate cover shown in FIG. 38.

FIG. 40 is a side view showing an example of a cover for a dish that is installed inside the incubator shown in FIG. 29.

FIG. 41 is a schematic structural view showing a third embodiment of the biological sample culturing and observation system according to the present invention.

FIG. 42 is a plan view of the incubator shown in FIG. 41 and shows a state in which a well plate is installed.

FIG. 43 is a cross-sectional view showing an example of a culturing vessel that is installed inside the incubator shown in FIG. 41.

FIG. 44 is a cross-sectional view showing another example of a culturing vessel that is installed inside the incubator shown in FIG. 41.

FIG. 45 is a cross-sectional view showing another example of a culturing vessel that is installed inside the incubator shown in FIG. 41.

FIG. 46 is a cross-sectional view showing another example of a culturing vessel that is installed inside the incubator shown in FIG. 41.

FIG. 47 is a cross-sectional view showing another example of a culturing vessel that is installed inside the incubator shown in FIG. 41.

FIG. 48 is a cross-sectional view showing another example of a culturing vessel that is installed inside the incubator shown in FIG. 41.

FIG. 49 is a cross-sectional view showing another example of a culturing vessel that is installed inside the incubator shown in FIG. 41.

FIG. 50 is a cross-sectional view showing another example of a culturing vessel that is installed inside the incubator shown in FIG. 41.

FIG. 51 is a cross-sectional view showing another example of a culturing vessel that is installed inside the incubator shown in FIG. 41.

FIG. 52 is a cross-sectional view showing another example of a culturing vessel that is installed inside the incubator shown in FIG. 41.

FIG. 53 is a cross-sectional view showing another example of a culturing vessel that is installed inside the incubator shown in FIG. 41.

FIG. 54 is a cross-sectional view showing another example of a culturing vessel that is installed inside the incubator shown in FIG. 41.

FIG. 55 is a cross-sectional view showing another example of a culturing vessel that is installed inside the incubator shown in FIG. 41.

FIG. 56 is a cross-sectional view showing another example of a culturing vessel that is installed inside the incubator shown in FIG. 41.

FIG. 57 is a cross-sectional view showing another example of a culturing vessel that is installed inside the incubator shown in FIG. 41.

FIG. 58 is a bottom view showing another example of a culturing vessel that is installed inside the incubator shown in FIG. 41.

FIG. 59 is a cross-sectional view showing another example of a culturing vessel that is installed inside the incubator shown in FIG. 41.

FIG. 60 is a cross-sectional view showing another example of a culturing vessel that is installed inside the incubator shown in FIG. 41.

FIG. 61A is a plan view showing another example of a bottom glass reinforcing member that is used in the culturing vessel shown in FIG. 57, while FIG. 61B is a cross-sectional view of this reinforcing member.

FIG. 62 is a cross-sectional view showing an example of a bottom glass reinforcing member that is used in the culturing vessel shown in FIG. 57.

FIG. 63 is a cross-sectional view showing an example of a bottom glass reinforcing structure that is used in the culturing vessel shown in FIG. 57.

FIG. 64 is a cross-sectional view showing another example of a bottom glass reinforcing structure that is used in the culturing vessel shown in FIG. 57.

FIG. 65 is a plan view showing another example of a bottom glass reinforcing member that is used in the culturing vessel shown in FIG. 57.

FIG. 66 is a plan view showing another example of a bottom glass reinforcing member that is used in the culturing vessel shown in FIG. 57.

FIG. 67 is a plan view showing another example of a bottom glass reinforcing member that is used in the culturing vessel shown in FIG. 57.

### DETAILED DESCRIPTION OF THE INVENTION

Embodiments of the biological sample culturing and observation system according to the present invention will now be described based on the drawings.

A biological sample culturing and observation system is formed so as to enable a variety of biological samples to be cultured in research fields such as biology, reproduction, and biotechnology, and so as to also enable the culture state of the biological samples to be observed. Note that, here, the term biological sample includes such as cells, ova, and biomedical tissue, or microorganisms such as algae, fungi, and bacteria and, in each of the embodiments described below, a case is described in which cells are cultured as an example of a biological sample.

### First Embodiment

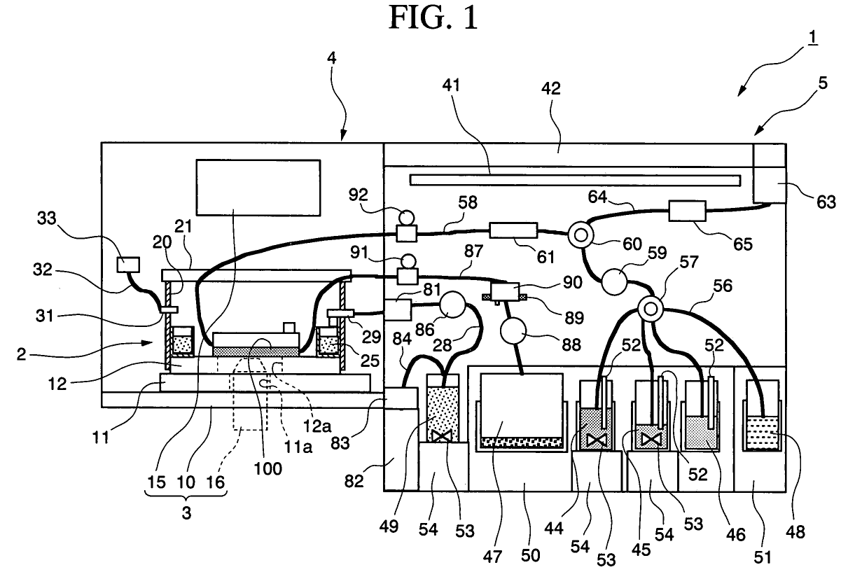

An example of the structure of the first embodiment of the biological sample culturing and observation system of the present invention will now be described based on FIGS. 1 to 28.

FIG. 1 is a schematic structural view of a biological sample culturing and observation system. This biological sample culturing and observation system 1 is used in order to culture a biological sample while observing temporal changes in the biological sample that is being cultured. The biological sample culturing and observation system 1 is provided with an incubator 2 that houses the biological sample that is being cultured such that it can be optically observed from the outside and that maintains its internal culturing environment in a state of isolation from the outside and in a predetermined condition. The biological sample culturing and observation system 1 is also provided with an observation optical system 3 that optically observes a biological sample from outside the incubator 2, a blocking device 4 that blocks external light that is irradiated on the biological sample or that is within the visual field of the observation optical system, and a supply device 5 that selectively supplies liquid or gas to a biological sample inside the incubator 2 from a plurality of reagent vessels (holding vessels) 44, 45, and 46 that hold a plurality of types of liquid or gas that are necessary for culturing or observation.

The incubator 2 is formed in a box shape that is fixed to a base 10 of the observation optical system 3 and is able to be opened and closed. In this embodiment, the base 10 is fixed, for example, on a Y axis movable stage 12. Moreover, in the same way as a commercially available incubator such as a CO₂ incubator, the incubator 2 performs temperature control and culture gas concentration control so as to create a high humidity environment.

A case will now be described in which a gas that includes carbon dioxide is used as one of the culture gases.

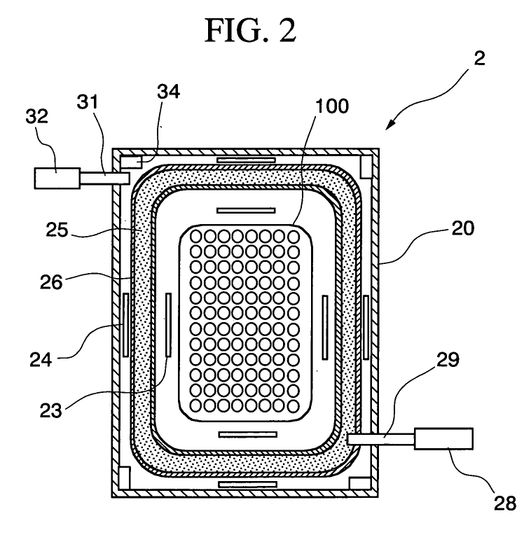

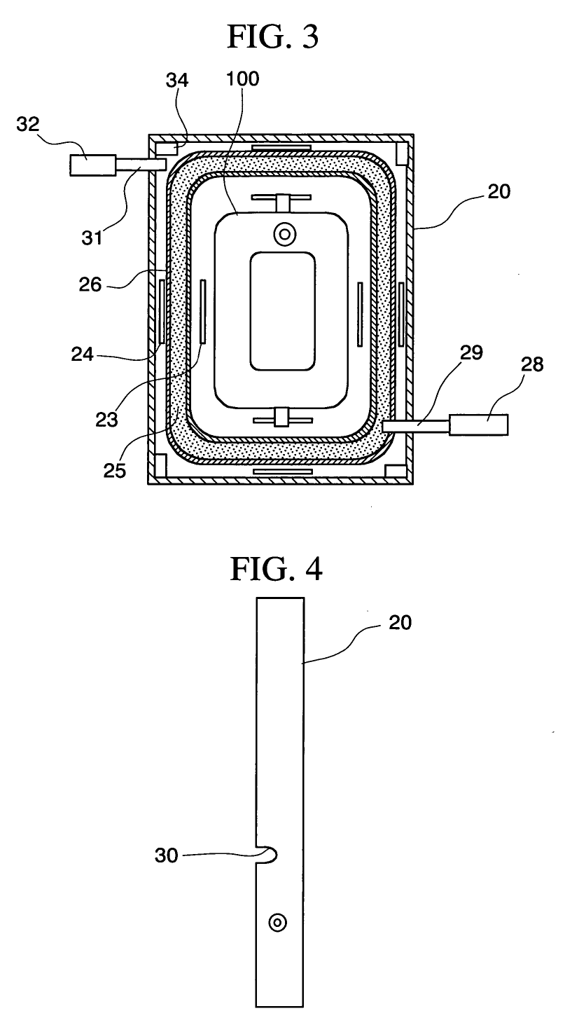

A culturing vessel that is open to the atmosphere (described below) is housed inside the incubator 2 as a biological sample culturing vessel 100. However, the incubator of the present invention is not limited to this and it is also possible to house a culturing vessel that is closed to the atmosphere or a dish, flask, or well plate. FIG. 2 shows an example in which a well plate is used as the culturing vessel 100, while FIG. 3 shows an example in which an atmospherically open type of dedicated culturing vessel 100 is used as the culturing vessel.

The incubator 2 is provided with a case 20 and a top cover 21 that closes an upper aperture of the case 20. The case 20 may be formed, for example, from alumite treated aluminum or highly corrosion resistant stainless steel. A material having a low coefficient of thermal conductivity is desirable. This is in order that temperature changes inside the case can be controlled.

The top cover 21 includes an optical glass member that has an anti-reflection coating provided on both surfaces thereof. Note that, the anti-reflection coating may be provided on one surface only. However, if transmission observation and irradiated illumination observation are to be performed simultaneously, then coating both surfaces is desirable. Moreover, it is desirable that the optical glass of the top cover 21 is of a size that allows transmitted illumination to be irradiated onto the entire surface of the culturing vessel.

The top cover 21 is not fixed to the case 20 of the incubator, but is able to be separated therefrom. The top cover 21 is fitted such that it can be opened and closed or can be removed in order to facilitate cleaning or replacement of an objective lens 16 or the culturing vessel. Note that it is also possible for the top cover 21 to be fixed using a required minimum quantity of screws or the like. Furthermore, it is desirable that the airtightness of the top cover 21 is improved using packing or the like (not shown). This is because, by improving the airtightness, it is possible to achieve stable environment control without the atmosphere inside the incubator 2 escaping to the outside.

As shown in FIG. 1, the Y axis movable stage 12 also functions as a bottom portion of the incubator 2. A miniature or tape-shaped heater (not shown) is mounted on the base 10. This heater is mounted at a position that enables it to uniformly heat the culturing vessel 100.

Miniature temperature sensors 24 that are used to control an auxiliary heater 26 are mounted at predetermined locations based on the relationship of a difference between the temperature of the interior of the incubator 2 and the temperature of the auxiliary heater 26. In addition, miniature temperature sensors 23 that measure the temperature of the culturing vessel 100 are mounted around the culturing vessel 100.

As shown in FIGS. 2 and 3, a water tank 25 that has a wide aperture area is also provided inside the incubator 2. This water tank 25 is preferably positioned symmetrically inside the incubator 2, and is still more preferably formed in a toroidal shape. As a result, carbon dioxide mixture gas that has been warmed by the auxiliary heater 26 and gasified circulates uniformly in a natural convection current in the culturing vessel.

In order to perform auxiliary heating of the water tank 25, the miniature or tape-shaped auxiliary heater 26 is mounted in contact with the water tank 25. In addition, a humidification water supply coupling 29 is provided that enables a humidification water supply tube 28 to be connected to a side surface of the case 20 of the incubator 2.

The auxiliary heater 26 accelerates the evaporation of water inside the water tank 25, and also minutely adjusts the temperature inside the incubator 2.

As shown in FIG. 4, a notch 30 is provided in a side surface of the case 20 of the incubator 2. This enables a waste solution discharge tube 87 and a common tube 58 for supplying culture solution supply that are connected to the atmospherically open culturing vessel 100 to be connected together, and the tubes 58 and 87 are able to be removed easily simply by removing the top cover 21 of the incubator 2. Furthermore, a sealing member (not shown) made of rubber or the like is attached to the perimeter of the notch 30 such that the airtight state is maintained when the top cover 21 is fitted, and the internal environment such as the temperature, the humidity, the carbon dioxide concentration and the like does not deteriorate.

A coupling 31 for a carbon dioxide sensor is provided on a side surface of the case 20 of the incubator 2. The concentration of carbon dioxide inside the incubator 2 can be measured from outside the box by a carbon dioxide sensor 33 that is provided at a distal end of a tube 32 that is connected to the carbon dioxide sensor coupling 31. As a result, it is possible to verify whether or not the concentration of carbon dioxide inside the incubator 2 has reached the concentration that is required to culture a biological sample.

Note that the measurement of the carbon dioxide concentration is not limited to being performed externally, and it is also possible for the carbon dioxide sensor 33 to be provided inside the incubator. It is even more desirable for a fan 34 to be mounted inside the incubator 2. The fan 34 causes the gasified carbon dioxide-humidified air mixture to move in a convection current inside the incubator 2 and disperses the humidified air such that the internal temperature in the incubator 2 is made uniform.

The fan 34 is desirably positioned so that air is not blown directly onto the culturing vessel 100 so that there is no localized cooling of the culturing vessel 100.

The observation optical system 3 may be formed, for example, by a microscope. The observation optical system 3 may be provided with additional members in the form of a base 10 that forms a stage portion on which the subject to be observed is mounted, and also, for example, an illumination optical system 15 that may include, for example, a transmitted illumination system that illuminates the subject being observed.

An example of the illumination optical system 15 is an IR illumination system that emits long wavelength illumination light, which has little effect on cells. It is also possible for the top cover 21 of the incubator 2 to be provided with the same properties as an IR filter, in which case the illumination optical system 15 does not need to be an IR illumination system.

The base 10 is provided with an X axis moveable stage 11 that moves reciprocally to the left and right in FIG. 1, and with the Y axis movable stage 12 that moves reciprocally towards and away from a person viewing FIG. 1. The stages are able to be moved electrically, and the incubator 2 that is fixed onto the Y axis movable stage 12 can be moved to a desired position within a predetermined range by a signal from a computer (not shown). The X axis movable stage 11 and the Y axis movable stage 12 are driven via a ball screw by a motor (not shown). The symbol 16 in FIG. 1 is an objective lens, which is one of the component members forming the observation optical system 3. The objective lens 16 is positioned such that cells inside the culturing vessel 100 can be observed via the base 10, and holes 11a and 12a of a predetermined size are provided in the X axis movable stage 11 and the Y axis movable stage 12.

It is also possible to insulate the objective lens 16 in order to prevent any drop in the temperature of the culturing vessel 100 or to prevent any change from occurring in the internal temperature of the incubator 2 as a result of the objective lens 16 being adjacent to the bottom of the culturing vessel 100.

For example, the objective lens 16 may be enclosed in a box-shaped insulating portion (not shown) and this insulating portion kept at, for example, 37° C. In this case, the insulating portion is kept at 37° C. However, because the objective lens 16 is isolated from the rest of the optical system by the insulating box, any thermal effects on the rest of the optical system can be reduced.

Another method involves directly wrapping a heater whose shape can be varied such as a tape heater around the objective lens 16. In this method, the structure to insulate the objective lens 16 can be simplified.

Another even more preferable method involves providing a partition so as to isolate the portion where the objective lens 16 is being insulated from the environment in which the cells are being cultured. By employing this type of structure, an atmosphere that is being temperature controlled can be prevented by the partition from escaping through the holes 11a and 12a that are provided in the X axial movable stage 11 and the Y axial movable stage 12. Furthermore, the need to perform complex temperature control of the insulating portion can be lessened. Moreover, even if the culture solution were to leak out due to the culturing vessel being broken or the like, the possibility of the observation optical system 3 becoming contaminated can be reduced.

Note that this partition may be formed by a hard plate-shaped member. However, it is desirably formed by a flexible or elastic sheet that is positioned so as to join a distal end of the objective lens 16 to the base 10 or the incubator 2.

If fluorescent observation of the cells is performed, then a structure may be employed in which, for example, an irradiated illumination system or the like (not shown) is provided and excitation light is irradiated from the cell adhesion surface side (the objective lens side). The light that is consequently generated from the cells is then condensed by the objective lens. If, on the other hand, the light emitted from the cells is to be observed, then an irradiated illumination system is not required. However, it is desirable that an image formation system having a high NA is used.

Furthermore, it is also possible to provide an image pickup device (not shown) and repeatedly pick up images of a cell at, for example, a constant time interval via the observation optical system 3. By then analyzing the picked-up images, the cell behavior can be measured.

The blocking device 4 is used to prevent external light from reaching the incubator 2. For example, a flat plate-shaped member may be formed from a metal or plastic blocking material using an appropriate bending process. The blocking device 4 also has an effect of blocking the incubator 2 from the outside environment so that it is possible to control any changes in the temperature inside the incubator 2 that are caused by changes in the outside environment.

Furthermore, the blocking device 4 is constructed such that it can be removed or such that it can be opened and closed in order that the interior of the incubator 2 can be accessed, and is mounted on the base 10.

It is also possible for a peephole to be provided in the blocking device 4 to allow to drop in at the culturing vessel 100. This peephole is provided so that an experimenter can, if necessary, directly observe a culture state. The peephole is closed by a lid when it is not required and is formed such that the internal portions of the blocking device 4 form a dark room.

A description will now be given of the supply device 5. An air cleaning unit 42 for purifying air that is formed by combining a bactericidal UV lamp 41 with a hepa filter and a fan is fixed to a frame ceiling portion of the supply device 5 in order to maintain a clean environment inside the supply device 5. This air cleaning unit is mounted so as to face the interior of the supply device 5.

Inside the supply device 5 are mounted reagent vessels 44, 45, and 46 that contain a culture solution or a reagent or a buffer solution such as PBS (−), a waste solution tank 47 in which old culture solution is stored, a reagent vessel 48 that contains reserve culture solution or reagent or buffer solution, a humidification water tank 49 in which is stored sterilized water for humidification, a refrigerator 50 that cold stores culture solution and waste solution, and a freezer 51 that preserves reagents that require freezing and also has a defrosting function. Each of these containers may be formed from a material that can be sterilized. Alternatively, a disposable structure may be employed in which the containers are formed using low cost resin or ordinary glass.

Liquid level sensors 52 that detect the surface of the culture solution or the like that is contained therein are mounted in the reagent vessels 44, 45, and 46. The liquid level sensors 52 are desirably a non-contact type in order that there is no contamination of the interior of the culturing vessel 100. However, a contact type of liquid level sensor may be used provided that a sterilization operation such as autoclave sterilization is possible. Note that it is also possible for no liquid level sensor to be provided, and for a structure to be employed in which an experimenter visually confirms the quantity of solution that remains.

Moreover, because it is possible for the liquid level sensor 52 to detect in advance any changes in the quantity of solution inside a reagent vessel by calculating the relationship between the supply and demand of culture solution, a constant optimum quantity of culture solution can be supplied and any problems such as leaking culture solution and the like can be detected early.

Agitators 53 are provided in the reagent vessels 44 and 45 and in the humidification water tank 49 in order to stir the solution contained therein. These agitators 53 are rotated by magnetic stirrers 54 that are located beneath the containers 44, 45, and 49. The agitators 53 are formed from a material that can be sterilized. Rotating the agitators 53 prevents any concentration distribution of a reagent and allows the delivery of a culture solution (reagent) in which the components are always contained uniformly.

Shakers may also be employed as a method of stirring the culture solution other than using a combination of the magnetic stirrers 54 and the agitators 43. In a shaker, the top plate performs an iterative operation in a circle, an oval, reciprocatingly, in a seesaw motion, or in a FIG. 8 so as to stir a liquid. If the stirrer uses a shaker, then there is no need to clean the agitators 53 and the possibility of contamination of the culture solution can be more easily avoided.

The stirring may be performed constantly. However, because it is sufficient if the solution components are uniform when the culture solution is being supplied, the stirring only needs to be performed prior to the delivery of the culture solution.

One end of respective culture solution supply tubes 56 that are used to supply culture solution, reagent, or buffer solution to the culture vessel 100 is connected to each of the vessels 44, 45, 46, and 48. The other end of each of these culture solution supply tubes 56 is connected to a flow path switching valve 57 that selects the reagent to be used. Note that the flow path switching valve 57 is provided with a spare port.

Such a spare port may be used, if required, to connect the culture solution supply tube from the freezer 50, or may be used to connect the culture solution supply tube from some other supply unit. It is thus possible to greatly expand the types of experiment that can be performed.

One common culture solution supply tube 58 that delivers all of the respective supplies from the flow path switching valve 57 is connected to the culturing vessel 100.

A culture solution supply pump 59, a flow path switching valve 60 and a warming tank 61 are installed along the common culture solution supply tube 58 in this sequence moving from the flow path switching valve 57 towards the culturing vessel 100.

Inside the supply device 5 are provided a clean air tank 63, an air tube 64 that extends from the clean air tank 63, and an air pump 65 that is installed on this air tube 64 in order to supply clean air to the interior of the common culture solution supply tube 58. A distal end of this air tube 64 is connected to the flow path switching valve 60. The fluid to flow along the common culture solution supply tube 58, which is connected to the culture vessel 100, can be selected using the flow path switching valve 60 from either a liquid such as the culture solutions that are stored in the reagent vessels 44, 45, 46, and 48 or the clean air that is stored in the clean air tank 63.

The warming tank 61 is used to warm the culture solution or clean air that is flowing inside the common culture solution supply tube 58. Note that the warming tank 61 does not need to be formed in a tank shape and it is also possible to employ a structure in which the tube itself is warmed.

The culture solution supply pump 59 may be a peristaltic pump and may control the supply and discharge of culture solution by drawing air into and out of a reagent vessel like an aspirator.

Another method is to use a chiller-warmer instead of the refrigerator 50, and perform chilling and warming at the same time. If this type of structure is employed, then the warming tank 61 can be omitted. Moreover, if a liquid or gas is supplied to the culturing vessel 100 after it has been held for a length of time in a warm area such as the incubator 2, then because the culture solution or the like has been warmed in this warm area, it is again possible to omit the warming tank 61 in the same way.

By warming a supplied liquid or gas using the warming tank 61 or some other method, it is possible to reduce any change in the temperature inside the culturing vessel 100 that may be due to the introduction of such liquids or gases.

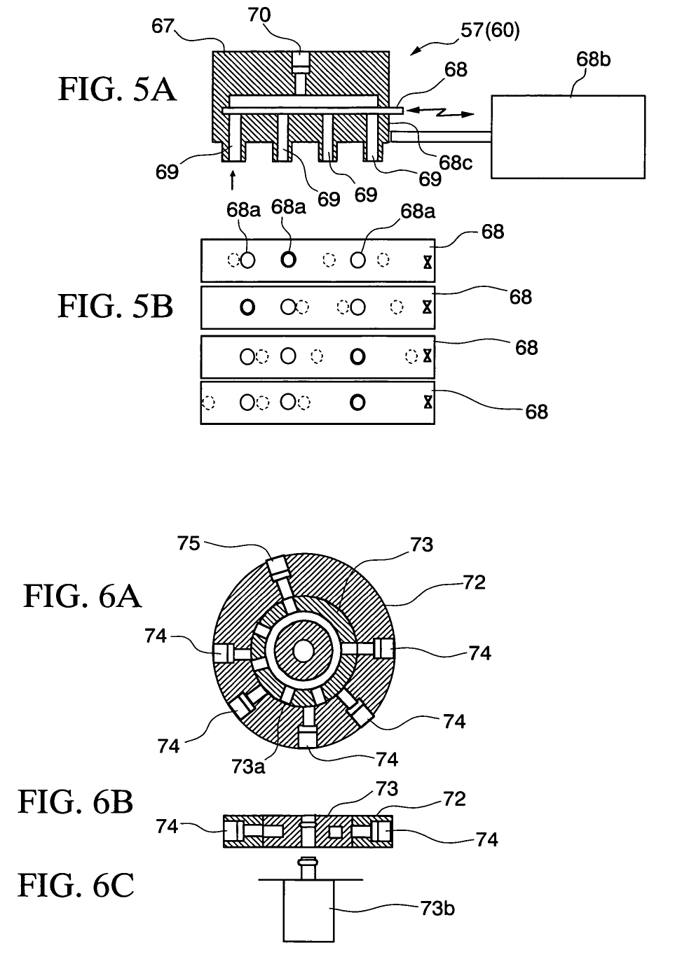

Examples of the flow path switching valves 57 and 60 shown in FIG. 1 are switching valves such as those shown in FIGS. 5A through 8B. As shown in FIGS. 5A and 5B, a plate-shaped valve body 68 that is located inside a valve box 67 is able to move in intervals of a predetermined distance in the longitudinal direction thereof. In addition, either one of or a plurality of a plurality of holes 68a that are provided in the valve body 68 engages with one of or a plurality of corresponding input ports 69 that are provided in the valve box 67. At this time, fluid is able to flow from the input ports 69 to an output port 70. The method used to perform this operation may be one in which, for example, the valve box 67 is placed at a predetermined position in the supply device 5, and a protrusion 68c that is provided on the valve body 68 is engaged in an anchoring groove (not shown) that is provided in an output shaft of a direct-action motor 68b. In this state, by operating the direct-action motor 68b, the valve body 68 moves in parallel therewith and the switching of the port 69 is performed automatically.

The flow path switching valves 57 and 60 are formed from a material that is capable of undergoing autoclave sterilization. Alternatively, if a disposable structure is employed, then it is possible to further lower the possibility that the cells will become contaminated.

In the flow path switching valve shown in FIGS. 6A to 6C, a rotary type of valve body 73 is provided inside a valve box 72. The valve body 73 is able to be rotated for predetermined angular distances by a motor 73b and either one of or a plurality of a plurality of holes 73a that are provided in the valve body 73 engages with one of or a plurality of corresponding input ports 74 that are provided in a side of the valve box 72. At this time, fluid is able to flow from the input ports 74 to an output port 75 provided in the valve box 72.

In the flow path switching valve shown in FIGS. 6A to 6C, the valve box is formed having a reasonably thickness so that the input ports 74 do not protrude to the outside. As a result, end surfaces of the pipe are located inside the switching valve, and any possibility of the culture solution or the like becoming contaminated is further reduced.

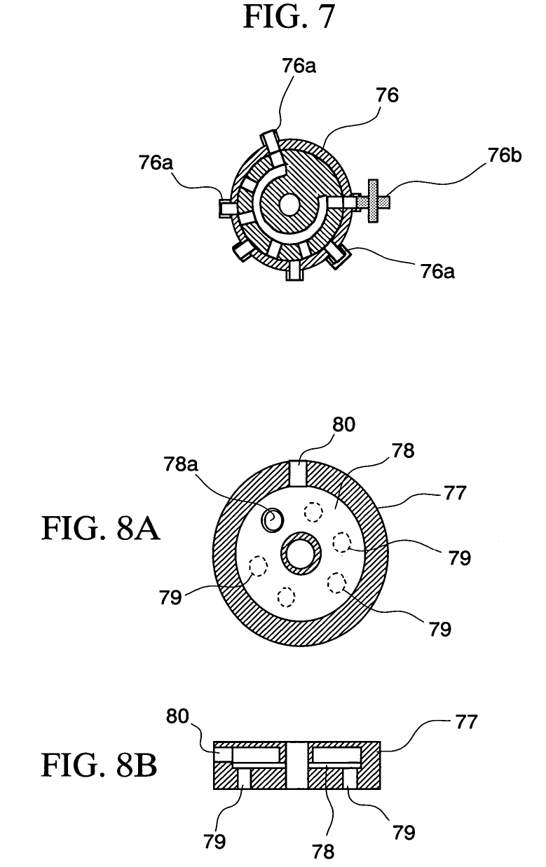

The flow path switching valve shown in FIG. 7 is the same as the switching valve shown in FIGS. 6A to 6C, and is a rotary type of flow path switching valve. In the flow path switching valve shown in FIG. 7, unlike the switching valve shown in FIGS. 6A to 6C, a valve box 76 is formed reasonably thinly so that input ports 76a and an output port 76b protrude to the outside. The size and weight of the valve box 76 can be reduced by an amount corresponding to this decreased thickness.

Furthermore, because a structure to fix the tubes does not need to be provided inside the flow path switching valves 57 and 60, the valve structures thereof can be simplified. As a result, cleaning is easy and these switching valves can reused after they have been cleaned and sterilized.

The flow path switching valve shown in FIGS. 8A and 8B is also a rotary type flow path switching valve like the switching valves shown in FIGS. 6A to 6C and FIG. 7. In the flow path switching valve shown in FIGS. 8A and 8B, a rotary type of circular plate-shaped valve body 78 is provided inside a valve box 77. The valve body 78 is able to be rotated for predetermined angular distances, and the one hole 78a that is provided in the valve body 78 engages with one of a plurality of input ports 79 that are provided in a bottom surface of the valve box 77. At this time, fluid is able to flow from the input port 79 to an output port 80 provided in the bottom surface of the valve box 77.

In each of the flow path switching valves shown in FIGS. 5A to 8B, the valve body can be moved automatically by a drive source such as a solenoid or motor. Accordingly, the valve body can be switched automatically by a remote operation without an operator needing to perform a manual operation.

The flow path switching valve can be broken down into a drive source such as a motor and a movable portion. The flow path can then be switched by connecting a movable portion, to which a tube is connected, to a drive source.

A carbon dioxide tank 82 that supplies carbon dioxide to the humidification water tank 49 and an air pump 83 are provided in the supply device 5. A carbon dioxide supply tube 84 that extends from the air pump 83 extends as far as the humidification water tank 49. Moreover, a humidification water supply tube 28 extends from the humidification water tank 49. A distal end of the humidification water supply tube 28 is connected to a humidification water supply coupling 29 on a side face of the incubator 2. A humidification water supply pump 86 that supplies humidification water from the humidification water tank 49 to the incubator 2 and a carbon dioxide humidification water warming tank 81 that is provided with an in-built heater and that warms the humidification water to a suitable temperature are each connected to the humidification water supply tube 28. The humidification water warming tank 81 may also be formed integrally with a portion that holds the humidification water tank 49.

If a suitable concentration of carbon dioxide gas is blown directly into the incubator 2, then a temperature distribution is created inside the incubator 2 by the flow motion of the gas. As a result, because, for example, there may be localized cooling of the culturing vessel 100, the environment inside the culturing vessel 100 is no longer uniform and the cell condition is different in each portion inside the culturing vessel 100. As a result, the reliability of results from observing cultured cells is deteriorated.

Therefore, if a structure is employed in which carbon dioxide is mixed into the humidification water, this carbon dioxide slowly gasifies from the surface of the humidification water. As a result, no temperature distribution is generated inside the culturing vessel and neither is there any concentration distribution of the carbon dioxide. Accordingly, it is possible to construct a more stable culturing environment.

Liquids (for example, culture solution) that are needed for culturing and gases (for example, culture gas) that are needed for culturing form nutrients for the biological sample being cultured. As is described above, a gas containing carbon dioxide is used as an example of a culture gas. However, gas containing oxygen or gas containing nitrogen can also be used as the culture gas.

A waste solution discharge tube 87 extends from the waste solution tank 47 to the incubator 2. A distal end of the waste solution discharge tube 87 is connected to the culturing vessel 100. A discharge pump 88 that discharges old culture solution from the culturing vessel 100 to the waste tank 47 and a liquid reservoir 90 that is provided with a light absorption meter 89 are respectively connected to the waste solution discharge tube 87.

Note that a spare port is provided in the liquid reservoir 90. By providing a spare port, it is possible to connect tubes from a larger number of liquid containers than the number of liquid containers that can be located inside the supply device 5. Accordingly, the system can be used in a wider variety of experimental methods.

For example, it is possible to connect tubes from other external units that hold bottles containing reagents requiring freezing and that have freezing and defrosting functions. As a result, it is possible to provide a function of defrosting these reagents immediately prior to their being supplied to the culturing vessel 100.

Flow meters 91 and 92 are connected respectively to the waste solution discharge tube 87 and the common culturing solution supply tube 58 directly in front of where these are inserted into the incubator 2 from the supply device 5. As a result, it is possible to measure the flow rate of the culture solution that is supplied to the culturing vessel 100 and the flow rate of the culture solution that is discharged from the culturing vessel 100.

As is described above, the culturing vessel 100 that is placed inside the incubator 2 may be a commercially available dish, flask, or well plate. It is also possible to use the culturing vessel that is open to the atmosphere that is described below.

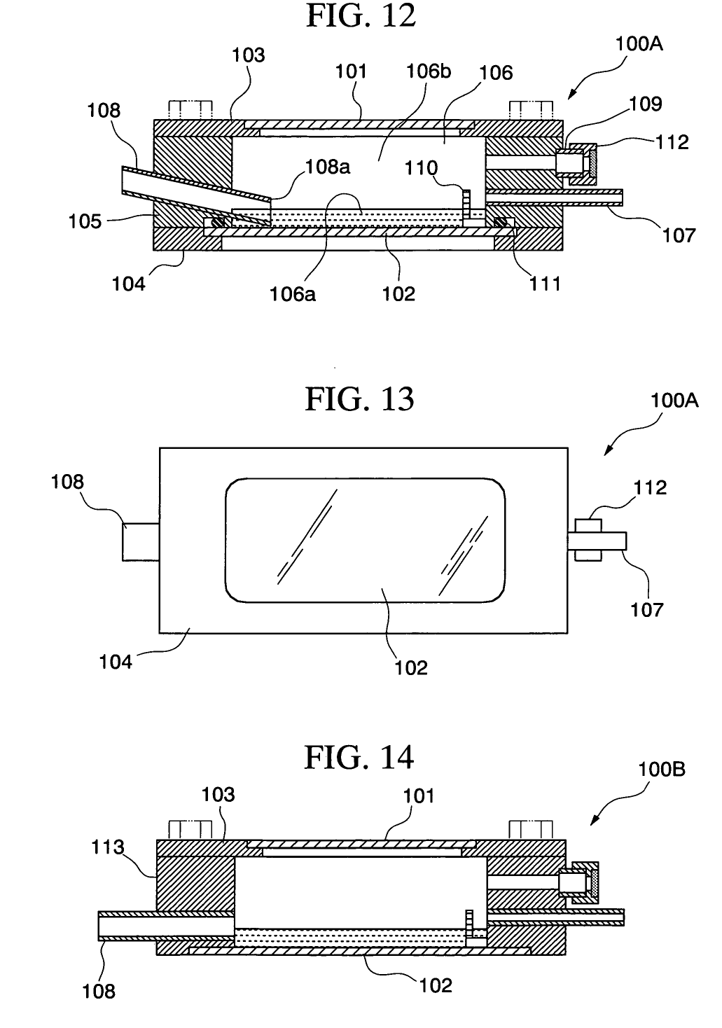

As shown in FIGS. 12 and 13, an atmospherically open type of culturing vessel 100A is provided with a top glass 101 and a bottom glass 102 that have optical characteristics and are positioned apart from each other in a vertical direction, a top glass fixing member 103 and a bottom glass fixing member 104 that respectively fix the top glass 101 and the bottom glass 102, and a central member 105 that is placed between the top glass fixing member 103 and the bottom glass fixing member 104 and forms side walls of the culturing vessel. A culturing chamber 106 is formed inside the space created by the top glass fixing member 103, the bottom glass fixing member 104, and the central member 105. In FIG. 13, the outer configuration of the culturing vessel 100A is a square configuration. However, it is not essential that the outer configuration thereof be a square configuration and, for example, the outer configuration may be a round configuration or a hexagonal configuration or the like.

The top glass 101 and the bottom glass 102 are used as transparent plates that allow the light required for an optical observation to pass through. In addition to the aforementioned glass plates, specific examples of a transparent plate may include transparent resin plates and the like that are formed from a material that is not toxic to a biological sample.

Namely, the culturing vessel 100 provided inside the incubator 2 has a culturing section that holds the biological sample being cultured, and a light transmitting section that transmits the light required to make an optical observation onto an optical path located between the observation optical system and the biological sample held in the culturing section.

The central member 105 is provided with a culture solution supply port 107 that is connected to the common culture solution supply tube 58 and guides culture solution that is supplied from the common culture solution supply tube 58 to the interior of the culturing vessel 100A, a waste solution discharge port 108 that is connected to the waste solution discharge tube 87 and discharges old culture solution from the waste solution discharge tube 87, a carbon dioxide-moisture mixture intake aperture 109 that is used to take a mixture of gasified carbon dioxide and moisture from the water tank 25 inside the incubator 2, and a commutation member 110 that adjusts the flow of culture solution.

The waste solution discharge port 108 is formed so as to be open at a position above the surface of the solution in FIG. 12. However, it can also be formed so as to be completely submerged in the solution.

Glass that has been AR coated in portions-corresponding to the surface on the culturing chamber 106 side or glass that has received a hydrophilic coating in portions corresponding to the surface on the culturing chamber 106 side may be used for the top glass 101. If AR coated glass is used, then the light transmission is improved and an excellent observation of a biological sample can be made based on this transmission and irradiated illumination.

A lattice member can be adhered onto, for example, the culturing chamber 106 side of the bottom glass 102 in order to provide reinforcement.

The top glass 101 is fixed to the top glass fixing member 103 by being inserted therein or by adhesion, while the bottom glass 102 is fixed to the bottom glass fixing member 104 by being inserted therein or by adhesion. The bottom glass fixing member 104 is fixed to the central member 105 such that the vessel is hermetically sealed via an O ring 111.

In the mutual relationship between the top glass fixing member 103 and bottom glass fixing member 104 and the central member 105, by employing, for example, an assembly structure that uses screws or a fit-together structure, it becomes possible to disassemble the top glass fixing member 103, the bottom glass fixing member 104, and the central member 105. Moreover, the respective members forming the culturing vessel 100A, namely, the top glass fixing member 103, the bottom glass fixing member 104, the central member 105, the culture solution supply port 107, and the commutation member 110 are made from a material that is not toxic to cells such as, for example, PEEK, PPS, or PSF.

The culturing chamber 106 is divided along a horizontal line into a culture solution layer 106a and a gas layer 106b. The culture solution supply port 107 is positioned below the commutation member 110 and above the surface of the culture solution layer 106a.

The waste solution discharge port 108 also functions as a gas discharge port for discharging the gas inside the culturing chamber 106. A culture solution suction portion 108a of the waste solution discharge port 108 is positioned slightly above the observation surface. The culture solution supply port 107 and the waste solution discharge port 108 can be blocked off by plugs such as rubber caps. Note that the structure of these plugs may be such that they are assembled in advance together with the culture solution supply port 107 and the waste solution discharge port 108, and the blocked off state of these apertures can be ended automatically when the culture solution supply tube 56 and the waste solution discharge tube 87 are connected respectively thereto.

The waste solution discharge port 108 is mounted at a predetermined angle of inclination such that an end portion of the waste solution discharge port 108 that is located on the external side of the culturing vessel 100A is higher than an end portion thereof that is located on the internal side of the culturing vessel 100A. In the same way as the waste solution discharge port 108, the culture solution supply port 107 may also be mounted at a predetermined angle of inclination such that an end portion of the culture solution supply port 107 that is located on the external side of the culturing vessel 100A is higher than an end portion thereof that is located on the internal side of the culturing vessel 100A.

Note that the culture solution supply port 107 and the waste solution discharge port 108 can also be used as cell suspension solution supply ports to supply cell suspension solutions using a pipette or the like. It is also possible to supply a cell suspension solution by removing the top glass fixing member 103.

A cap 112 that is provided with an anti-bacterial filter is attached to the carbon dioxide-moisture mixture intake aperture 109. In this case, by slightly loosening the cap 112 in order to provide ventilation, ventilation of the culturing vessel 100A can be performed easily. It is also possible for the carbon dioxide-moisture mixture intake aperture 109 to be used as a cell suspension solution supply port to supply a cell suspension solution using a pipette or the like.

Note that the carbon dioxide-moisture mixture intake aperture 109 is provided in the central member 105 in FIG. 12. However, the present invention is not limited to this and this aperture may also be provided, for example, in the top glass fixing member 103.

The commutation member 110 has a lattice configuration in which are formed a plurality of gaps that are made by constructing grooves or columnar protrusions having a depth or height of, for example, 0.5 to 1 mm. Note that it is not essential for the gaps to be formed at equal intervals in the commutation member 110. For example, it is also possible for the gaps to be provided densely in the vicinity of the culture solution supply port 107 and for the gaps to be provided sparsely on both sides of the culture solution supply port 107.

Other examples of an atmospherically open type of culturing vessel will now be described. As shown in FIG. 14, the top glass 101 of the culturing vessel 100B is fixed to the top glass fixing member 103 by being inserted therein or by adhesion. A common central member 113 that is formed by integrating the bottom glass fixing member with the central member is provided in the culturing vessel 10B. The bottom glass 102 is adhered to a bottom portion of the common central member 113 so as to hermetically seal the vessel. Moreover, the waste solution discharge port 108 is not tilted and is substantially parallel to the top glass 101 and the bottom glass 102.

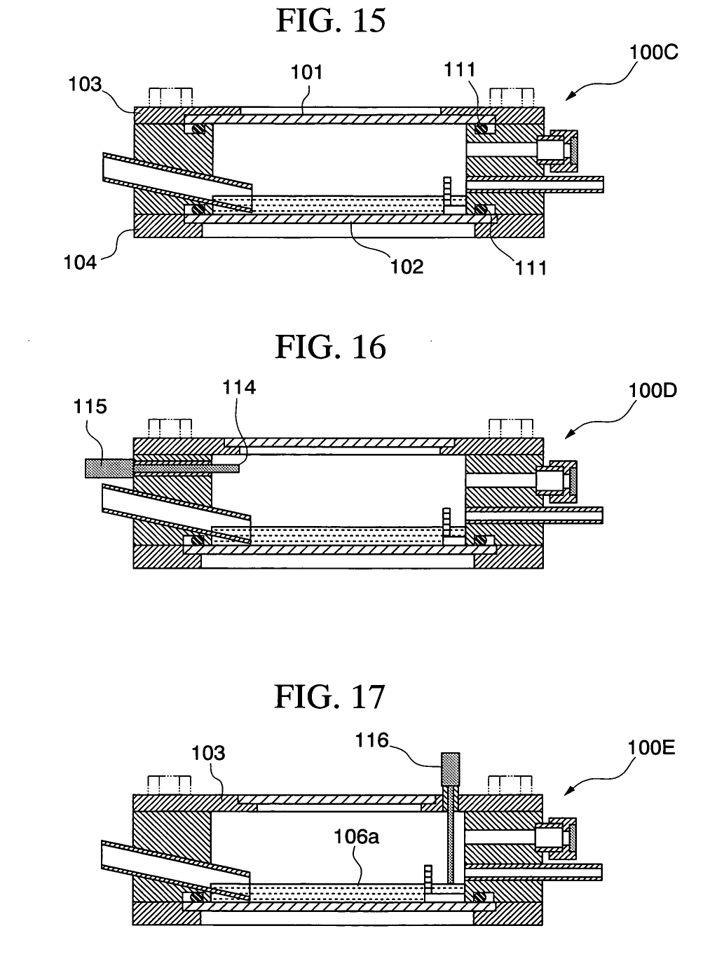

As shown in FIG. 15, the top glass fixing member 103 and the bottom glass fixing member 104 are provided in the culturing vessel 100C. The top glass fixing member 103 and the bottom glass fixing member 104 are attached to the central member 105 via an O ring 111 (specifically, an O ring 111 that is placed against both the top glass 101 and the bottom glass 102) such that the vessel is hermetically sealed. The top glass 101 is fixed to the top glass fixing member 103 by being inserted therein or by adhesion. The bottom glass 102 is fixed to the bottom glass fixing member 104 by being inserted therein or by adhesion.

As shown in FIG. 16, a culturing vessel 100D is provided with a carbon dioxide supply coupling 114 that guides carbon dioxide inside the culturing vessel 100D to the outside of the vessel, and a carbon dioxide sensor 115 that is mounted on an external end of the carbon dioxide supply coupling 114. The carbon dioxide sensor 115 makes it possible to detect the concentration of carbon dioxide inside the culturing vessel.

As shown in FIG. 17, a liquid level sensor 116 is mounted on the top glass fixing member 103 of a culturing vessel 100E. The liquid level sensor 116 makes it possible to detect the height of the surface of the culture solution layer 106a inside the culturing chamber 106.

As shown in FIG. 18, the carbon dioxide supply coupling 114, the carbon dioxide sensor 115, and the liquid level sensor 116 are mounted on a culturing vessel 100F.

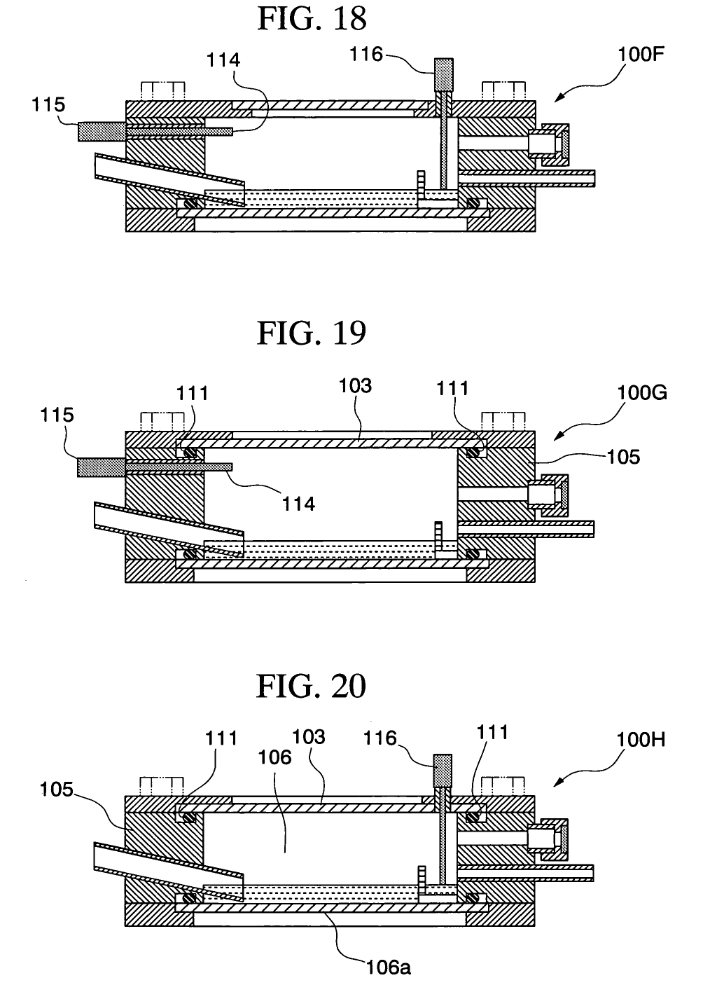

As shown in FIG. 19, the top glass fixing member 103 is attached to the central member 105 of a culturing vessel 100G via an O ring 111 such that the vessel is hermetically sealed. Furthermore, the carbon dioxide supply coupling 114 and the carbon dioxide sensor 115 are provided in the culturing vessel 100G so that the concentration of carbon dioxide inside the culturing vessel can be detected.

As shown in FIG. 20, the top glass fixing member 103 is attached to the central member 105 of a culturing vessel 100H via an O ring 111 such that the vessel is hermetically sealed. Furthermore, the liquid level sensor 116 is provided in the culturing vessel 100H so that the height of the surface of the culture solution layer 106a inside the culturing chamber 106 can be detected.

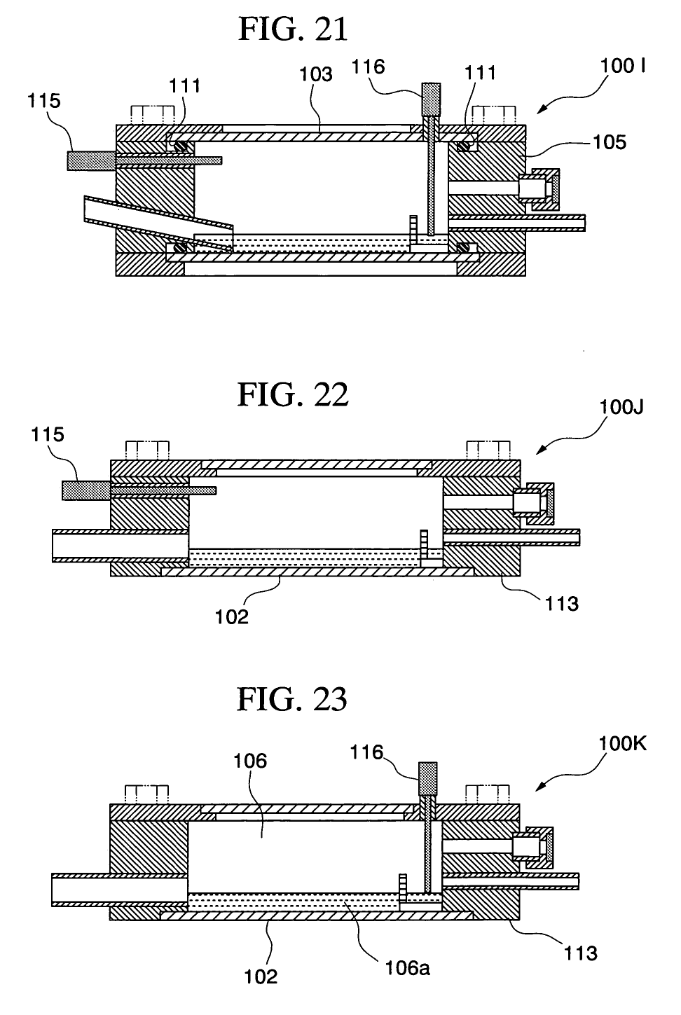

As shown in FIG. 21, the top glass fixing member 103 is attached to the central member 105 of a culturing vessel 100I via an O ring 111 such that the vessel is hermetically sealed. Furthermore, the carbon dioxide sensor 115 and the liquid level sensor 116 are provided in the culturing vessel 100I.

As shown in FIG. 22, a common central member 113 that is formed by integrating the bottom glass fixing member with the central member is provided in a culturing vessel 100J. The bottom glass 102 is adhered to a bottom portion of the common central member 113 so as to hermetically seal the vessel. Furthermore, the carbon dioxide sensor 115 is provided in the culturing vessel 100J so that the concentration of carbon dioxide inside the culturing vessel can be detected.

As shown in FIG. 23, a common central member 113 that is formed by integrating the bottom glass fixing member with the central member is provided in a culturing vessel 100K. The bottom glass 102 is adhered to a bottom portion of the common central member 113 so as to hermetically seal the vessel. Furthermore, the liquid level sensor 116 is provided in the culturing vessel 100K so that the height of the surface of the culture solution layer 106a inside the culturing chamber 106 can be detected.

As shown in FIG. 24, a common central member 113 that is formed by integrating the bottom glass fixing member with the central member is provided in a culturing vessel 100L. The bottom glass 102 is adhered to a bottom portion of the common central member 113 so as to hermetically seal the vessel. Moreover, the carbon dioxide sensor 115 is provided in the culturing vessel 100L so that the concentration of carbon dioxide inside the culturing vessel can be detected. Furthermore, the liquid level sensor 116 is provided in the culturing vessel 100L so that the height of the surface of the culture solution layer 106a inside the culturing chamber 106 can be detected.

Note that drive systems of the X axis movable stage 11 and the Y axis movable stage 12, which are component members of the observation optical system 3, are each electrically connected to a computer, and are automatically controlled as is appropriate by command signals from the computer.

Moreover, the auxiliary heater 26, the temperature sensors 23 and 24, and the carbon dioxide sensor 33 that are located inside the incubator 2 as well as the liquid level sensors 52, the flow path switching valves 57 and 60, the air pumps 65 and 83, the humidification water supply pump 86, and the waste solution discharge pump 88 that are assembled in the supply device 5 are electrically connected to a computer, and are controlled comprehensively by command signals from this computer.

Next, an operation of a biological sample culturing and observation system that is constructed in the manner described above will be described.

Firstly, as shown in FIG. 1, the culturing vessel 100 is set inside the incubator 2, and the water tank 25 is filled with sterilized water. A heater (not shown) is then controlled based on signals from the temperature sensor 23 so that the temperature inside the incubator 2 is kept within a predetermined range. Moreover, the humidity inside the incubator 2 is kept with a predetermined range while the temperature of the sterilized water inside the water tank 25 is controlled by controlling the auxiliary heater 26 based on signals from the temperature sensors 24 that are provided adjacent to the water tank 25.

The control of the heater that is not shown in the drawings may be ON/OFF control or proportional control. However, if temperature control is performed using PID control or cascade control or the like, then the temperature can be controlled with a greater degree of accuracy.

In the same way, the control of the auxiliary heater 26 may be performed using PID control or cascade control or the like when the efficiency of the heat transfer to the vicinity of the culturing vessel is good. As a result, it is possible to control the effects of thermal shock on cells that is the result of heat transmission from the heater warming the water tank.

The concentration of carbon dioxide inside the culturing vessel is controlled in the following manner. Namely, the air pump 83 is driven by signals from the carbon dioxide sensor 33, and carbon dioxide inside the carbon dioxide tank 82 is supplied via the carbon dioxide supply tube 84 to the humidification water tank 49. In addition, carbon dioxide is mixed into the sterilized water inside the humidification water tank 49. The humidification water supply pump 86 is then driven and sterilized water into which carbon dioxide has been mixed is supplied via the humidification water supply tube 28 to the water tank 25 inside the incubator 2. Because the water tank 25 is warmed to an appropriate temperature by the auxiliary heater 26, as is described above, water gas is supplied constantly from here to the interior of the incubator 2 and the optimum humidity for culturing cells is maintained. At this time, the carbon dioxide that has been mixed into the sterilized water end the water tank 25 is also supplied.

Furthermore, by performing control such that the temperature of the water tank 25 is higher than the temperature of the humidification water tank 49, it is possible to accelerate the discharge of the carbon dioxide into the air. Note that the concentration of carbon dioxide in the carbon dioxide tank 82 is not limited to 100%, and, for example, it may be mixed with air to a concentration of approximately 5%. It is also possible for a waterproof transparent film to be provided on a top opening portion of the water tank 25.

Because sterilized water for humidification in which carbon dioxide has been mixed is able to be supplied from the outside of the incubator 2 via the humidification water supply tube 28, it is possible to supply the optimum quantity of humidification water and carbon dioxide constantly to the interior of the incubator 2.

Moreover, it is possible to supply culture solution and reagent to the culturing vessel 100 by switching the flow path switching valves 57 and 60 and by using the ON/OFF operation of the air pump 65. The culture solution and reagent can also be supplied in a mixed state.

Namely, if, for example, a stimulus is to be imparted to a biological sample by a reagent, then a liquid obtained by mixing in advance the appropriate reagent in a culture solution or the like is injected, for example, into a reagent vessel 44. By then switching the flow path switching valve 57 so that it is connected to the reagent vessel 44, the culture solution supply pump 59 is driven. As a result, culture solution in which the reagent has been mixed can be supplied directly to the culturing vessel 100 via the common culture solution supply tube 58 by the culture solution supply pump 59.

At the same time as this, the discharge pump 88 is driven and the culture solution inside the culturing vessel is discharged via the waste solution discharge tube 87 to the waste solution tank 47. Accordingly, it is possible to keep the liquid inside the culturing vessel substantially at a constant level.

Another method of imparting a stimulus to a biological sample using a reagent involves switching the flow path switching valve 57 so that, for example, the reagent vessel 44 that holds culture solution is connected to the common culture solution supply tube 58. By then driving the culture solution supply pump 59, culture solution can be stored in advance in the warming tank 61. Next, by switching the flow path switching valve 57, another reagent vessel 45 that holds a reagent is connected to the common culture solution supply tube 58. By then driving the culture solution supply pump 59, the reagent is supplied to the interior of the warming tank 61, and the reagent is dissolved in the culture solution. The culture solution in which the reagent has been dissolved in this manner can then be supplied from the warming tank 61 to the culturing vessel 100 via the common culture solution supply tube 58.

As a result, because the temperature of the culture solution coming into the culturing vessel 100 is approximately the same as the temperature of the culturing environment, the temperature inside the culturing vessel 100 can be stabilized.

If all of the culture solution inside the culturing vessel is disposed of and a new reagent is introduced, firstly, only the waste solution discharge pump 88 is driven so that all of the culture solution is discharged into the waste solution tank 47 via the waste solution discharge tube 87. At the same time as this or slightly after this, the common culture solution supply pump 59 can be driven so that the flow path switching valve 57 is appropriately switched and culture solution or reagent is supplied to the culturing vessel via the common culture solution supply tube 58.

Moreover, if the structure of the device makes it possible to remove old culture solution inside the culturing vessel before culture solution in which reagent has been mixed reaches the culturing vessel 100, then the supply and discharge can be performed simultaneously by driving the pump 88 at the same time as the old culture solution is discharged. As a result, the time taken for supply and discharge can be shortened, and the possibility that the biological sample will dry out is decreased.

Furthermore, if an experimenter wishes to completely remove an administered reagent, then a buffer solution is injected into at least one of the plurality of reagent vessels 44, 45, 46, and 48, and, as is described above, after the culture solution inside the culturing vessel has firstly been removed, the flow path switching valve 57 is switched so that culture solution is supplied via the common culture solution supply tube 58 to the interior of the culturing vessel. As a result, the common culture solution supply tube 58 and the culture solution supply pump 59 are washed. Subsequently, the flow path switching valve 57 is again switched and culture solution is again supplied to the culturing vessel. Accordingly, any contamination between reagents can be prevented.

One method of supplying culture solution to the culturing vessel 100 is, for example, if the volume of the culturing vessel is 100 ml, to continuously drip the culture solution at a rate of approximately 140 μl per hour. An equivalent volume can be discharged at the same timing.

By replacing the culture solution at a rate of 140 μl per hour, for example, in three days 10 ml of culture solution can be totally replaced. Discharged culture solution can be disposed of in the waste solution tank 47.

Another method of supplying culture solution is what is known as a batch method. In this method, either half or all of the culture solution in the culturing vessel 100 is regularly disposed of after a predetermined number of days, and a predetermined quantity of new culture solution is supplied to replace this.

It is to be understood that this replacement quantity is not limited to half or all of the culturing solution, and an experimenter may designate a desired quantity to be replaced.

By employing this biological sample culturing and observation system, either a continuous method or a batch method can be freely selected.

Moreover, if a flow is switched using the flow path switching valve 57 shown in FIGS. 5 through 8, the solution in any one of the reagent vessels 44, 45, 46, and 48 can be supplied selectively to the culturing vessel 100. In addition, because the flow is switched using the flow path switching valve 57, the solutions in each of the reagent vessels 44, 45, 46, and 48 can be supplied to the culturing vessel using the single culture solution supply pump 59. This enables costs to be reduced compared with when a dedicated pump is provided for each reagent vessel.

When culture solution or reagent to be supplied to the culturing vessel 100 is changed by switching the flow path switching valve 57 as is described above, on each occasion the flow path switching valve 60 is switched and clean air that is stored in the clean air tank 63 is supplied to the common culture solution supply tube 58 by driving the air pump 65. When this switch is made, because the common culture solution supply tube 58 is filled with air, it is possible to prevent any more than the required volume of culture solution from being supplied to the interior of the culturing vessel 100. Moreover, by also supplying air when culture solution is being supplied or replaced, it is possible to adjust the flow of culture solution to the culturing vessel 100.

Furthermore, in this biological sample culturing and observation system, it is possible to use a control device to set different culture solution replacement periods depending on the type of cell that is the biological sample being observed. A computer, for example, may be used for the control device and by making an appropriate selection from a table that has been previously input into the computer, it is possible to set a culturing solution replacement period that provides the optimum culturing conditions for the cells that are the biological sample to be observed. Because of this, the number of steps where an experimenter must use personal judgment can be reduced, and observations can be made in identical culturing environments regardless of the experimenter.

Another method is to shift the focal point from the cells during observation and focus on the culturing solution so that the natural fluorescence inside the culture solution is detected. This can then be used to determine the culture solution replacement period.

Moreover, another method involves detecting the degree of optical absorption in the culture solution that is detected by the light absorption meter 89 that is provided in the liquid reservoir 90, and to determine the next culture solution replacement period from the relationship between the degree of optical absorption and the elapsed time since the previous replacement.

In this manner, it is possible to selectively set a variety of culturing environments and provide the interior of a culturing vessel with an ideal culturing environment without placing any stress thereon such as contamination or failures in the culture solution or reagent. At the same time as this, it is possible to observe a biological sample in this ideal culturing environment in real time.

Namely, using the blocking device 4 it is possible to observe a biological sample using the observation optical system 3, which may be a microscope or the like, while blocking external light from being irradiated onto the biological sample and into the field of vision of the observation optical system.

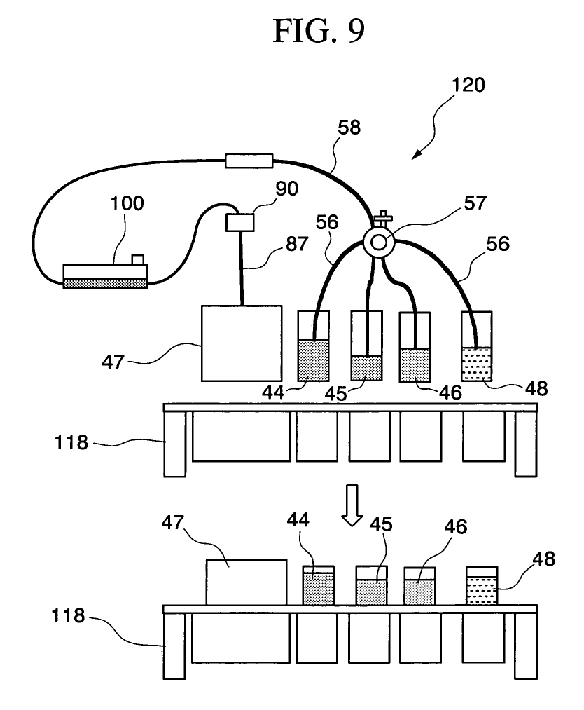

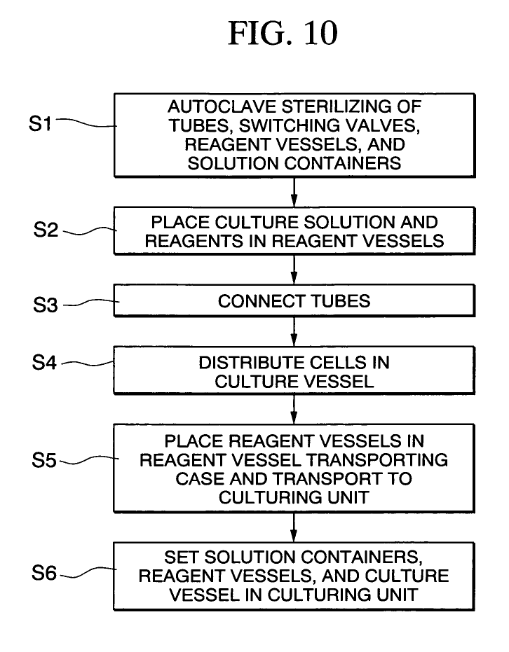

A description will now be given with reference made to FIGS. 9 and 10 of an example in which a culturing flow path system 120 is incorporated into the supply device 5.

Firstly, autoclave sterilization is performed on the culture solution supply tube 56, the common culture solution supply tube 58, the waste solution discharge tube 87, the flow path switching valve 57, the reagent vessels 44, 45, 46, and 48, the waste solution tank 47, and the liquid reservoir 90 (step S1). Next, if necessary, culture solution and various reagents are poured into the respective reagent vessels 44, 45, 46, and 48 within a clean bench (step S2). The respective tubes 56, 58, and 87 are then connected so as to form flow paths (step S3). Cells are then distributed in the culturing vessel 100 (step S4). The respective reagent vessels 44, 45, 46, and 48 are then placed inside a reagent vessel transporting case 118 and transported to the supply device 5 (step S5). The flow path switching valve 57, the reagent vessels 44, 45, 46, and 48, the waste solution tank 47, and the liquid reservoir 90 are then set in predetermined locations within the frame of the supply device 5 (step S6).

In this manner, the culturing flow path system 120 that is made up of the common culture solution supply tube 58, the waste solution discharge tube 87, the flow path switching valve 57, and the reagent vessels 44, 45, 46, and 48 is established in advance within a clean bench. It is, accordingly, possible using the reagent vessel transporting case 118 to simultaneously transport a flow path that is made up of the culturing vessel 100 and the respective tubes together with the reagent vessels 44, 45, 46, and 48. As a result, this flow path can be easily set in the biological sample culturing and observation system. Moreover, because this flow path can be set without needing to be touched by an operator, any contamination thereof can be prevented.

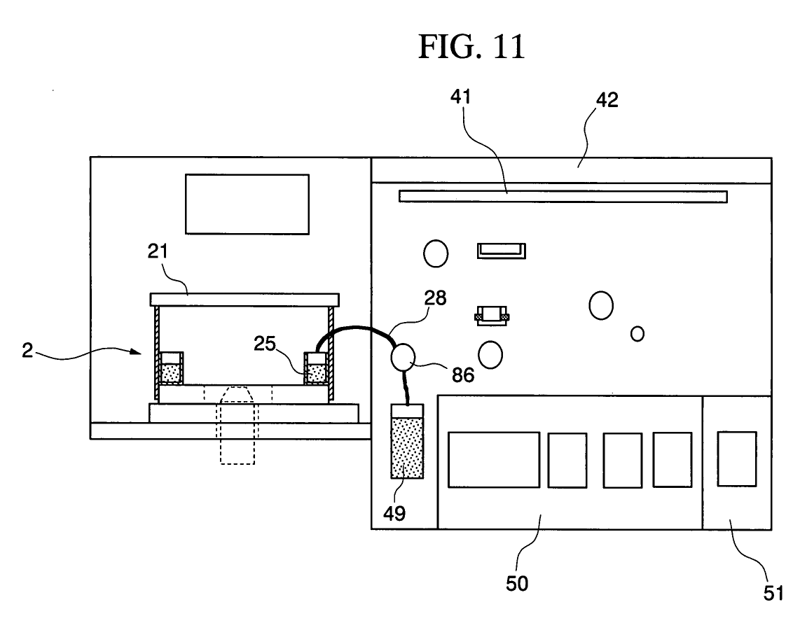

As shown in FIG. 11, because it is possible to begin humidification and warming of the interior of the incubator 2 prior to the culturing flow path system 120 that was established within the clean bench being set in the system, any observation that includes an experiment can proceed smoothly.

Moreover, because the flow meters 91 and 92 (see FIG. 1) are mounted respectively on the common culture solution supply tube 58 and the waste solution discharge tube 87, the flow rates inside these tubes 58 and 87 can be detected. Accordingly, the flow rate in the tubes 58 and 87 can be adjusted to one that is beneficial for the cells.

Because it is possible to confirm using the flow meters 91 and 92 that culture solution is flowing at a predetermined rate in conjunction with the operation of the pumps 59 and 88, it is also possible to detect such phenomena as breakages or blockages in a tube, or as a tube coming off one of the tube connecting portions or the like.

Moreover, because it is possible to control fluid quantities by providing the liquid level sensors 52 and 116 in the culturing vessel 100 and the respective reagent vessels 44, 45, 46, and 48, it is possible to provide advance warning of abnormalities such as leaks.

In addition, because it is possible by providing a spare port in the flow path switching valve 57 to connect additional culture solution and reagent vessels to the flow path switching valve 57, an even greater variety of combinations becomes possible so that a variety of experiments and observations can be handled.

Furthermore, because the light absorption meter 89 is provided in the liquid reservoir 90 on the waste solution discharge tube 87, it is possible to detect natural fluorescence or light absorption in discharged culture solution. As a result, a replacement timing can be detected from the relationship between the degree of optical absorption and the length of time since the last replacement, so that replacement can be performed automatically. The further effect is also obtained that variations in pH and the like that are caused by replacing the culture solution can be kept to a minimum.

Moreover, because the supply device 5 is provided with the refrigerator 50 and the freezer 51, the storage of reagents by refrigeration or freezing becomes possible. By then warming or defrosting these prior to use, deterioration of the reagents can be suppressed so that observations and experiments can be made using reagents that are always fresh.

In addition, because an anti-reflective AR coating is provided on the inner surface side, namely, on the culturing chamber 106 side of the top glass 101 of the culturing vessel 100, an improvement in transmittance is achieved.

Furthermore, because a hydrophilic coating is provided on the inner surface side of the top glass 101 of the culturing vessel 100 the effect is achieved that water droplets and the like can be prevented from adhering thereto.

Moreover, because the waste solution discharge port 108 of the culturing vessel 100 is inclined diagonally such that an outer side thereof, which is the side that connects to the tube, faces upwards, it is possible to prevent culture solution from running out during an assembly task on a clean bench or when it is being set up in the vessel. The culture solution supply port 107 may also be tilted such that an outer side thereof slopes upwards.

In addition, because volumes of solution can be controlled by providing the liquid level sensor 116 in the culturing vessel 100, advance warning of faults such as leaks and the like can be given.

Furthermore, by providing the carbon dioxide sensor 115 in the culturing vessel 100, the concentration of carbon dioxide inside the culturing chamber 106 can be monitored and feedback therefrom can be provided to the carbon dioxide supply side. As a result, the optimum concentration of carbon dioxide can be set constantly in the culturing chamber 106.

If a structure is employed in which a cap made out of rubber or the like is provided for the culture solution supply port 107 and the waste solution discharge port 108 of the culturing vessel 100, then it is possible to place the cap over the aperture that is not in use. Consequently, this device can also be used for incubation using a commercially available incubator and the like.

Moreover, because the respective apertures 107, 108, and 109 are used as cell suspension solution supply ports, cells and the like can be implanted in the culturing vessel 100 without the culturing vessel 100 needing to be disassembled.

In addition, by continuously supplying a minute quantity of culture solution, an atmospherically open type of culturing vessel is able to culture cells in a constantly fixed environment and is, therefore, effective for protocols where external factors need to be totally excluded. Furthermore, because the culture solution flow rate is extremely slow, this device can be used for cells that have weak shearing strength and for cells that have weak adhesive force. Moreover, if culture solution is supplied intermittently (for example, once every three days), cells can be cultured in the same state as in a commercial CO₂ incubator, which is effective for protocols that are performed in a constant culturing state.

Another example of a culture solution supply method is a circulating method. If a supply of culture solution is circulated to cells, then any changes in the environment are gradual. This is effective for protocols where external factors are to be excluded as far as possible.

In the supply device 5, by creating a down flow using a cleaning device (a hepa filter) and a fan, and installing the UV lamp 41 so as to provide an environment that is analogous to a clean bench, work can be performed in a substantially sterile environment even when reagents are added hastily after the start of culturing and observation.

### Variant Example of the First Embodiment

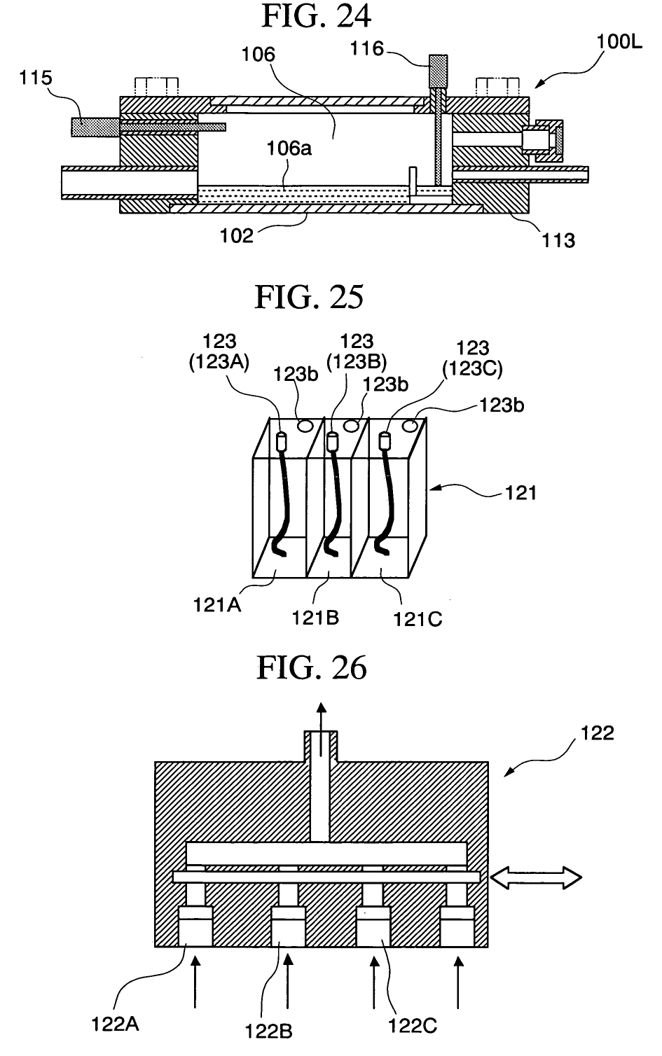

In the above description, the reagent vessels 44, 45, 46, and 48 are independent vessels. However, instead of this, it is also possible to employ, for example, a cassette-type tank 121 such as shown in FIG. 25. In this case, when selecting a reagent, a flow path switching valve 122 that switches the flow path using a mechanism such as a motor is used, as shown in FIG. 26, and an air-tight connection can be achieved by inserting couplings 123 (123A, 123B, and 123C) of the respective tanks 121A, 121B, and 121C that come out from the cassette tank 121 in the respective connection ports 122A, 122B, and 122C of the flow path switching valve 122.

By employing this type of structure, it is not necessary to connect any tubes so that the task of assembling the device within a clean bench is simplified. Furthermore, there is no possibility that the end of a tube will come into contact with an experimenter so that it is also possible to prevent any contamination that is due to carelessness when using solutions needed for culturing.

Note that the supplying of reagents to the interior of the cassette tank 121 may be performed via the coupling 123, or by using cylinders or the like installed via an opening portion 123b that is provided with a filter and can be opened and closed.

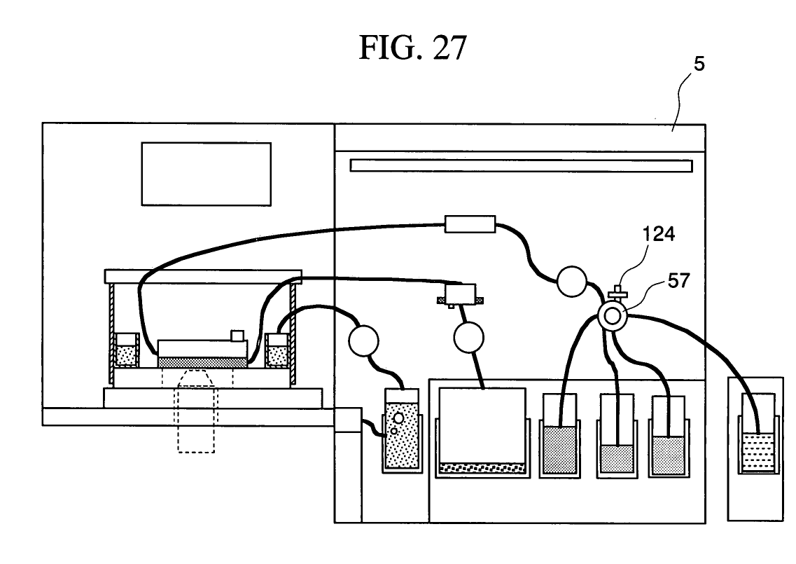

As is shown, for example, in FIG. 27, the structure of the flow path may be one in which an anti-bacterial filter 124 is fitted to the flow path switching valve 57 when the interior of the supply device 5 is a clean environment. This enables clean air to be introduced into the flow path via the anti-bacterial filter 124.

By employing this type of structure, the air pump 65 and flow path switching valve 60 shown in FIG. 1 become redundant. Accordingly, the structure is simplified and the tasks of connecting the tubes can be omitted. This also allows the cost of the device to be reduced.

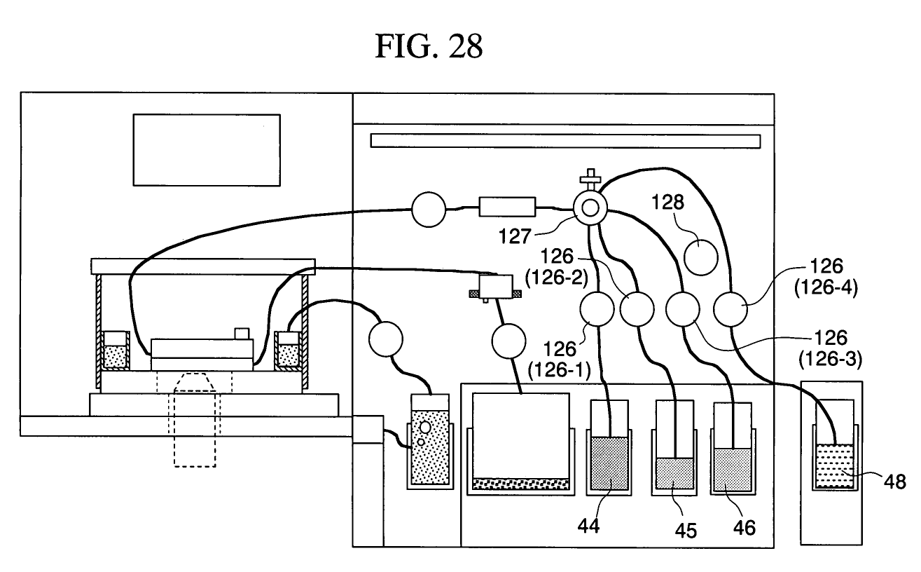

As is shown, for example, in FIG. 28, the structure of the flow path may be one in which there is provided a solution supply pump 126 (126-1, 126-2, 126-3, and 126-4) for each of the reagent vessels 44, 45, 46, and 48 and also a manifold 127 that has a plurality of tube connecting apertures. The supply of culture solution and reagent can then be freely set by causing the desired solution supply pump 126 (126-1, 126-2, 126-3, and 126-4) to operate.

By employing the above described structure, the flow path switching valves 57 and 60 in FIG. 1 are made redundant, and the structure is simpler compared with when the flow path is switched using a flow path switching valve. Accordingly, cleaning and sterilization are simplified, and preparations for experiments and observations can be performed easily. Moreover, if a structure is employed in which a reserve pump 128 is provided, then supply can be achieved simply by adding culture solution and reagent vessels, thereby enabling the device to be used in a variety of experiments.

Because a plurality of reagent vessels are provided, then, in addition to culture solution and PBS, reagents can also be supplied by operating the flow switching pump. It therefore becomes possible to measure changes and the like that are the result of cell stimulation, so that a wider variety of experiments can be performed.

Moreover, because selective supply is possible, discharging old culture solution, washing the interior of the flow path system by supplying PBS, and then supplying new solution become possible. As a result, long-term culturing observation becomes possible.

### Second Embodiment

A description of the second embodiment of the biological sample culturing and observation system of the present invention will now be given. Note that, in order to simplify the description, the same descriptive symbols are used for component elements that are the same as those used in the first embodiment and a description thereof is omitted.

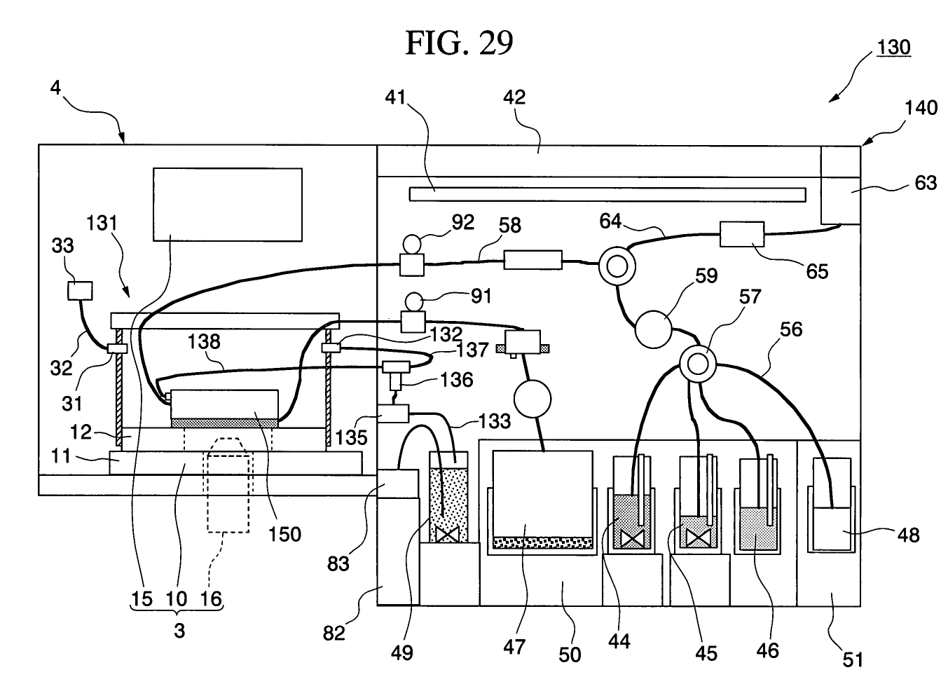

FIG. 29 is a schematic structural view of the biological sample culturing and observation system of the second embodiment. In an incubator 131 that is used in this biological sample culturing and observation system 130, a coupling 132 that is used to supply humidified air in which carbon dioxide has been mixed is provided at a side surface of the incubator 131. A carbon dioxide-humidified air mixture supply tube 137 that supplies the carbon dioxide-humidified air mixture to the interior of the incubator 131 is able to be connected to the carbon dioxide-humidified air mixture supply coupling 132. In conjunction with this, the water tank 25, the humidification water supply coupling 29, and the auxiliary heater that was used to warm the humidification water inside the water tank that were provided in the first embodiment have been eliminated.

A supply device 140 that is used in the second embodiment will now be described with reference made to FIG. 29. In the supply device 140, a carbon dioxide-humidified air mixture supply tube 133 extends from an upper gas layer portion of the humidification water tank 49. A heater 135 is provided partway along this carbon dioxide-humidified air mixture supply tube 133.

Furthermore, a flow path switching valve 136 is provided at a distal end of the carbon dioxide-humidified air mixture supply tube 133, and a carbon dioxide-humidified air mixture supply tube 137 and a carbon dioxide-humidified air mixture supply tube 138 extend from the flow path switching valve 136. The carbon dioxide-humidified air mixture supply tube 137 is connected to the carbon dioxide-humidified air mixture coupling 132, while the carbon dioxide-humidified air mixture supply tube 138 is connected to a culturing vessel 150 (described below).

Namely, either the incubator 131 or the culturing vessel 150 is selected by switching the flow path switching valve 136, and a carbon dioxide-humidified air mixture that has been mixed with humidification water in the humidification water tank 49 and gasified is then supplied to the selected destination.

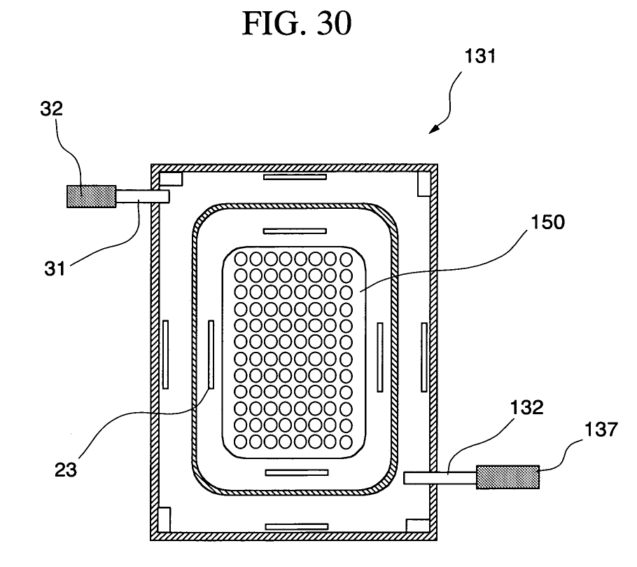

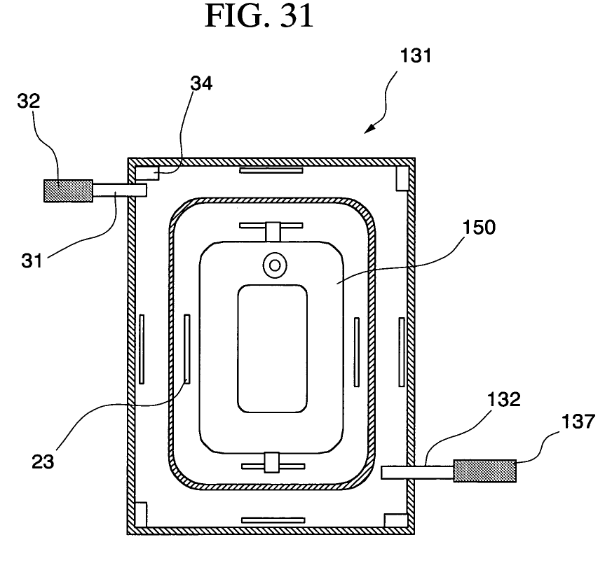

FIG. 30 shows an example in which a well plate is used as the culturing vessel 150, while FIG. 31 shows an example in which an atmospherically open type of dedicated culturing vessel is used for the culturing vessel 150.

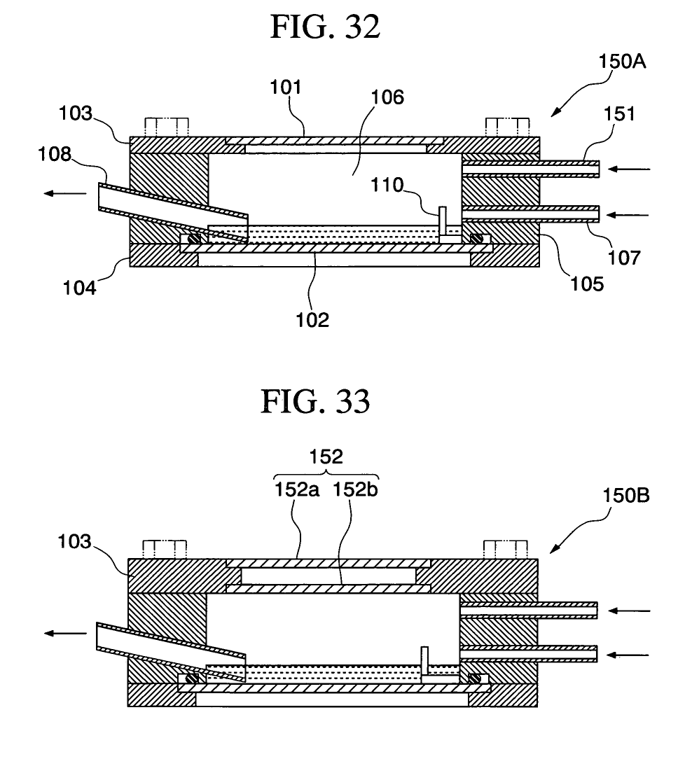

The culturing vessel that is used in the second embodiment will now be described. An atmospherically open type of culturing vessel 150A that is shown in FIG. 32 is provided with a top glass 101 and a bottom glass 102 that have optical characteristics and are positioned apart from each other in a vertical direction, a top glass fixing member 103 and a bottom glass fixing member 104 that respectively fix the top glass 101 and the bottom glass 102, and a central member 105 that is placed between the top glass fixing member 103 and the bottom glass fixing member 104 and forms side walls of the culturing vessel. A culturing chamber 106 is formed inside the culturing vessel 150A.

Note that the top glass fixing member 103 may be constructed such that it can be separated by undoing screws or the like, as shown in the drawing, or it may be adhered to or formed integrally with the central member 105. The bottom glass fixing member 104 may have the same structure as the top glass fixing member 103.

The central member 105 is provided with a culture solution supply port 107 that is connected to the culture solution supply tube 56 and guides culture solution that is supplied from the culture solution supply tube 56 to the interior of the culturing vessel, a waste solution discharge port 108 that is connected to the waste solution discharge tube 87 and discharges old culture solution from the waste solution discharge tube 87, and a commutation member 110 that adjusts the flow of culture solution. The waste solution discharge port 108 opens at a position above the surface of the culture solution.

Unlike the first embodiment, in the present embodiment, the waste solution discharge port 108 must be open at a position above the surface of the solution.

The reason for this is that, in the first embodiment, because culture gas is replaced via a filter, the pressure inside the culturing vessel can be maintained at atmospheric pressure even if the waste solution discharge port is positioned below the surface of the solution. However, in the present embodiment, because humidified air in which carbon dioxide has been mixed is supplied by a pump, it is necessary to expel the air inside the culturing vessel 150A to the outside in order to prevent any increase in the pressure inside the culturing vessel.

In this culturing vessel 150A, a carbon dioxide-humidified air mixture intake aperture 151 that is connected to the carbon dioxide-humidified air mixture supply tube 138 and takes in a carbon dioxide-humidified air mixture directly from there is provided in the central member 105.

Namely, the carbon dioxide-humidified air mixture intake aperture 151 is changed from a cap type to a tube connection type in order that a warmed carbon dioxide-humidified air mixture can be forcibly acquired.

It is preferable that the position of the carbon dioxide-humidified air mixture intake aperture 151 on the culturing chamber 105 side is as close as possible to the top glass 101.

As shown in FIG. 33, the top glass 152 that is mounted on the top glass fixing member 103 of a culturing vessel 150B has what is known as a pair glass structure that is formed by a pair of glass plates 152a and 152b that are mounted respectively on the top surface and the bottom surface of the top glass fixing member 103.

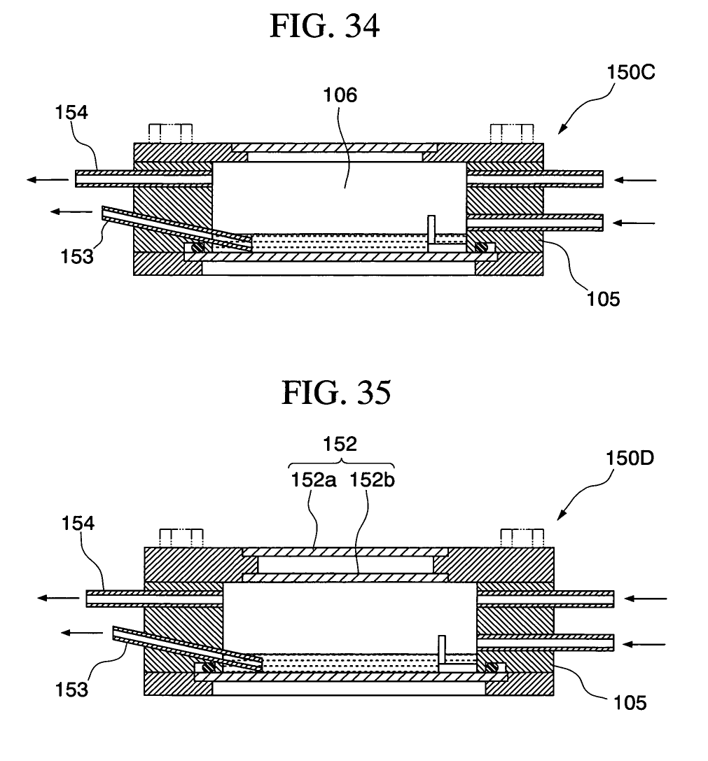

As shown in FIG. 34, a waste solution discharge port 153 and an air discharge port 154 are provided separately in the central member 105 of a culturing vessel 150C. Namely, old culture solution is discharged to the outside of the culturing chamber 106 via the waste solution discharge tube 87 that is connected to the waste solution discharge port 153, while the air inside the culturing chamber 106 is discharged to the outside through the air discharge port 154.

The diameter of the waste solution discharge port 153 is smaller than that of the waste solution discharge port 108 that is used in the culturing vessel 150A (see FIG. 32) and simultaneously discharges both air and waste solution. Moreover, the air discharge port 154 is located in the top portion of the culturing chamber 106. It is also possible for the cap 109 that was used in the culturing vessel in the first embodiment to be provided for the air discharge port.

As shown in FIG. 35, the top glass 152 of a culturing vessel 150D has what is known as a pair glass structure that is formed by the pair of glass plates 152a and 152b. Moreover, the waste solution discharge port 153 and the air discharge port 154 are provided separately in the central member 105 of the culturing vessel 150D.

Note that, in the same way as in the first embodiment, it is also possible for commercially produced dishes, flasks, and well plates to be used as the culturing vessel in addition to the above described atmospherically open culturing vessels 150A to 150D.

Another example involves constructing a culturing vessel by attaching a dedicated cover to a container such as a well plate or dish that is intended to contain a biological sample. In the description below, an example is given of a culturing vessel that is formed by attaching a dedicated cover to a well plate.

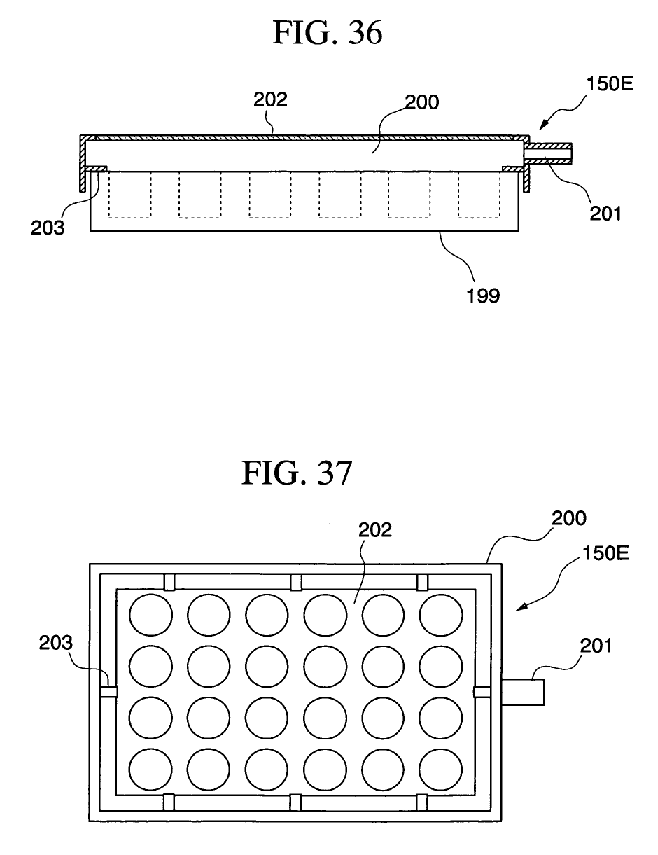

As shown in FIGS. 36 and 37, in a culturing vessel 150E, it is possible to supply culture gas by using a dedicated plate cover 200 on a commercial well plate 199. Namely, a culture gas supply coupling 201 is provided on the rectangular plate cover 200 that is formed such that it completely covers the well plate 199. The plate cover 200 is formed from transparent or colored resin or the like, and optically transparent glass 202 is attached above the well. In addition, a gas discharge space support portion 203 is provided on the plate cover 200 so as to protrude inwards. As a result of this gas discharge space support portion 203 being in contact with the top surface of the plate cover 200, a space for discharging gas is secured between the well plate 199 and the inner surface of the top plate of the plate cover 200. As shown in FIG. 37, a plurality of the gas discharge space support portions 203 are formed on inner surfaces of side wall portions of the plate cover 200 so as to protrude inwards.

Because culture gas that is fed via the culture gas supply coupling 201 is discharged through the gas discharge space formed between the well plate 199 and the inner surface of the top plate of the plate cover 200, atmospheric pressure can be constantly maintained inside the well plate 199.

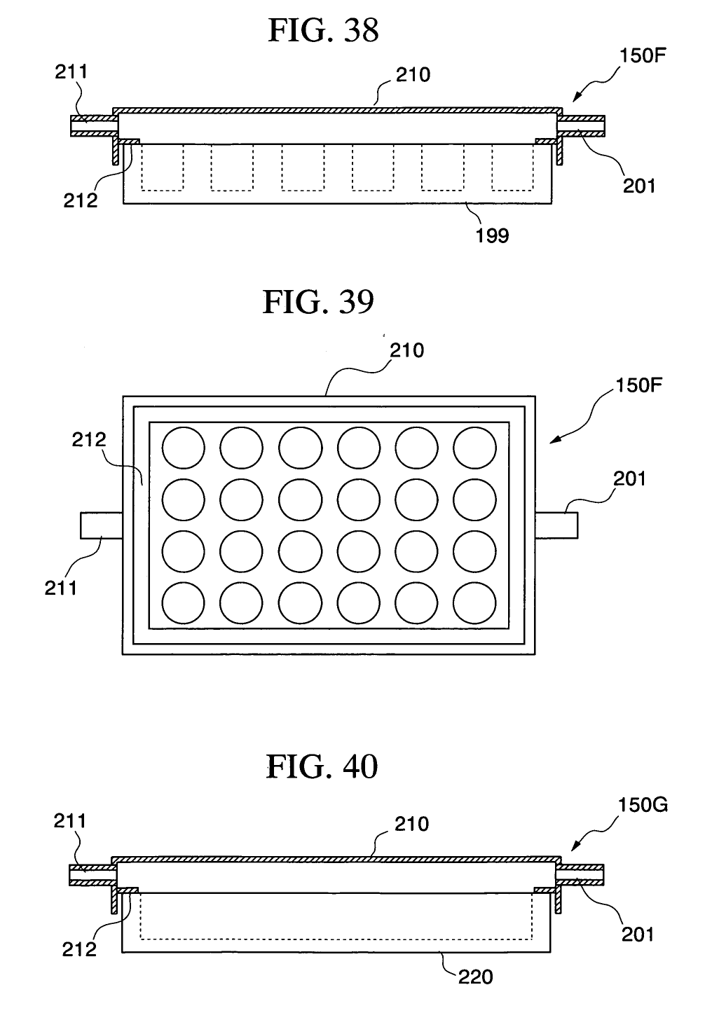

As shown in FIGS. 38 and 39, a dedicated plate cover 210 for the commercially available well plate 199 is also used for a culturing vessel 150F. In the culturing vessel 150F, the plate cover 210 does not have any glass, and is formed from transparent resin. Moreover, in addition to the culture gas supply coupling 201, a culture gas discharge coupling 211 is also provided on the plate cover 210. A cover support portion 212 is also provided not just in certain locations but around the entire periphery of the inner surface of the side wall portions of the plate cover 210. The interior of the culturing vessel 150F is shut off from the outside by this cover support portion 212.

According to this culturing vessel 150F, the chance of contamination can be further reduced by inserting a sterilized filter in the culture gas supply coupling 201 and the culture gas discharge coupling 211.

As shown in FIG. 40, a dedicated plate cover 210 for a commercially available dish 220 is used for a culturing vessel 150G In this culturing vessel 150G the same plate cover as the plate cover 210 used for the culturing vessel 150F shown in FIGS. 38 and 39 is used. In the culturing vessel 150G, instead of the commercially available well plate 199, the commercially available dish 220 is used for the vessel body. In this culturing vessel 150G, the chance of contamination can be further reduced by inserting a sterilized filter in the culture gas supply coupling 201 and the culture gas discharge coupling 211.

A description of the operation of the biological sample culturing and observation system having the above described structure will now be given with reference made to FIG. 29.

Carbon dioxide that has been stored in the carbon dioxide tank 82 is fed to the humidification water tank 49 by the air pump 83. In the humidification water tank 49, a carbon dioxide-humidified air mixture that contains a predetermined quantity of carbon dioxide is generated. This carbon dioxide-humidified air mixture is fed to the flow path switching valve 136 via the carbon dioxide-humidified air mixture supply tube 133. Partway along, the carbon dioxide-humidified air mixture is warmed to a suitable temperature by the heater 135. Thereafter, by switching the flow path using the flow path switching valve 136, the warmed carbon dioxide-humidified air mixture is supplied selectively to either the incubator 131 or the culturing vessel 150.

As a result, by warming the carbon dioxide-humidified air mixture using the heater 135, the occurrence of water droplets can be suppressed. In addition, it is possible to prevent cold air from flowing into the culturing vessel 150A and cooling the interior of the culturing vessel 150A.

Namely, as shown in FIG. 30, when a commercially available dish or well plate or the like is placed unmodified inside the incubator 131, the carbon dioxide-humidified air mixture is supplied to the interior of the incubator 131 by the carbon dioxide-humidified air mixture supply tube 137. Moreover, as shown in FIG. 31, if a dedicated culturing vessel such as that shown in FIGS. 32 through 35 or a dish 220 or well plate 199 such as those shown in FIGS. 36 through 40 are placed inside the incubator 131, then the carbon dioxide-humidified air mixture is supplied directly to the culturing vessel 150 via the carbon dioxide-humidified air mixture supply tube 138.

Because a carbon dioxide-humidified air mixture that is necessary for cell culturing and is obtained on the supply device 140 side can be supplied to the incubator 131 and directly to the culturing vessel 150, the water tank inside the incubator 131 is made redundant, thereby enabling the size of the incubator 131 to be reduced by a corresponding amount. Moreover, because it is no longer necessary to clean the water tank, the labor required to use this water tank is no longer needed.

Furthermore, by supplying a warmed carbon dioxide-humidified air mixture to the vicinity of the top glass 101 of the culturing vessel 150A and 150C when this warmed carbon dioxide-humidified air mixture is forcibly taken into the culturing chamber 106, as is shown, for example, in FIG. 32 and FIG. 34, it is possible to warm the top glass 101 and prevent condensation from forming on the top glass 101. As a result, in a phase contrast observation or the like that uses transmitted illumination, an image that has excellent contrast can be acquired.

Furthermore, if a pair glass structure is employed for the top glass 152, as is the case with the culturing vessels 150B and 150D shown in FIGS. 33 and 35, then any temperature changes in the culturing chamber can be suppressed as a result of the improved thermal insulation effect. Moreover, because it is possible to prevent condensation from forming on the top glass 152 itself, it is possible to perform culturing in which the effects of temperature change on the cells are suppressed. In addition, in a phase contrast observation or the like that uses transmitted illumination, an image that has excellent contrast can be acquired.

### Third Embodiment

A description of the third embodiment of the biological sample culturing and observation system of the present invention will now be given. Note that, in order to simplify the description, the same descriptive symbols are used for component elements that are the same as those used in the first embodiment and a description thereof is omitted.

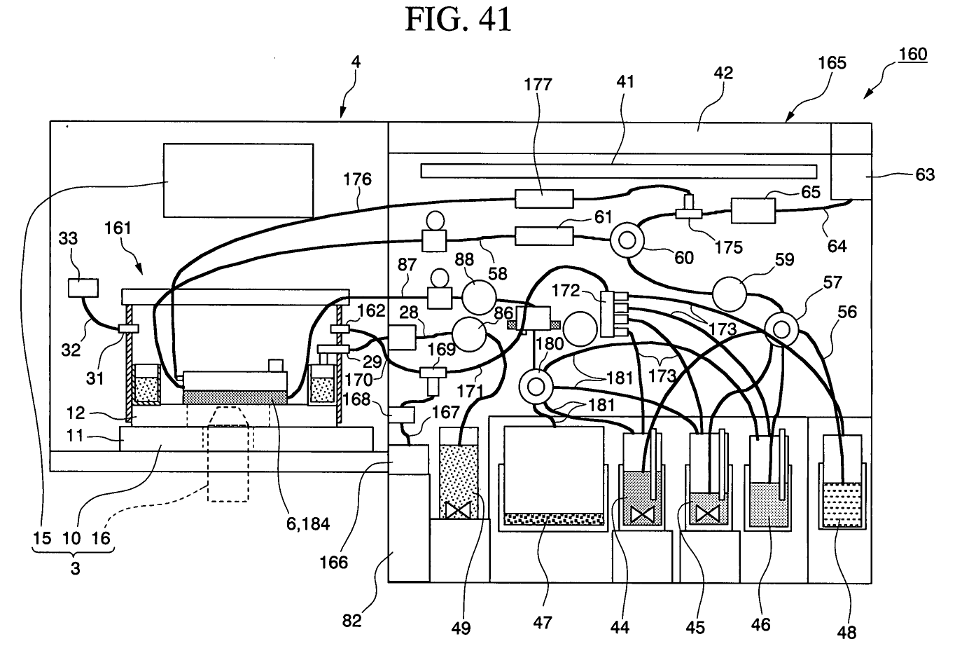

FIG. 41 is a schematic structural view of the biological sample culturing and observation system of the third embodiment.

In an incubator 161 that is used in this biological sample culturing and observation system 160, a carbon dioxide supply coupling 162 is provided on a side surface of the incubator 161. A carbon dioxide supply tube 170 that supplies carbon dioxide to the interior of the incubator 161 is able to be connected to the carbon dioxide supply coupling 162.

A supply device 165 that is used in the third embodiment will now be described. In the supply device 165, a carbon dioxide supply tube 167 extends from an air pump 166 that is attached to the carbon dioxide tank 82, and this carbon dioxide supply tube 167 is connected to a flow path switching valve 169 via an intermediate heater 168. Two carbon dioxide supply tubes 170 and 171 extend from the flow path switching valve 169. The carbon dioxide supply tube 170 is connected to the carbon dioxide supply coupling 162, while the carbon dioxide supply tube 171 is connected to the flow path switching valve 172.

A plurality of carbon dioxide supply tubes 173 extend from the flow path switching valve 172, and distal ends of each of these carbon dioxide supply tubes 173 are connected to the respective reagent vessels 44, 45, 46, and 48.

Namely, it is possible to select using the flow path switching valve 169 whether to supply carbon dioxide that is introduced via the air pump 166 from the carbon dioxide tank 82 to the incubator 161 side or to the reagent vessels 44, 45, 46, and 48 side. If supplying the reagent vessel side is selected, then a selection is made as to which of the plurality of reagent vessels 44, 45, 46, and 48 is to be supplied using the flow path switching valve 172.

An air switching valve 175 is provided on the air tube 64 that extends from the clean air tank 63, and a warm air supply tube 176 that is connected to the culturing vessel extends from this air switching valve 175. A heater 177 that warms the clean air inside the warm air supply tube 176 is provided on the warm air supply tube 176. Namely, it is possible to select using the air switching valve 175 whether to supply the clean air that is introduced via the air pump 65 from the clean air tank 63 to the common culture solution supply tube 58 side or to the warm air supply tube 176 side.

A flow path switching valve 180 is provided on the waste solution discharge tube 87. A plurality of waste solution discharge tubes 181 extend from the flow path switching valve 180, and distal ends of each of these waste solution discharge tubes 181 are connected to the waste solution tank 47 and the respective reagent vessels 44, 45, 46, and 48. As a result, if a sealable type of container is used as the culturing vessel, a circulatory system can be used to supply culture solution instead of the affusion system that is employed when an atmospherically open type of culturing vessel is used.

The culturing vessel used in the third embodiment will now be described. In the same way as in the first and second embodiments, a commercially available dish, flask, or well plate can be used for the culturing vessel. However, the sealed culturing vessel 190 and the atmospherically open culturing vessel 184 described below can also be used.

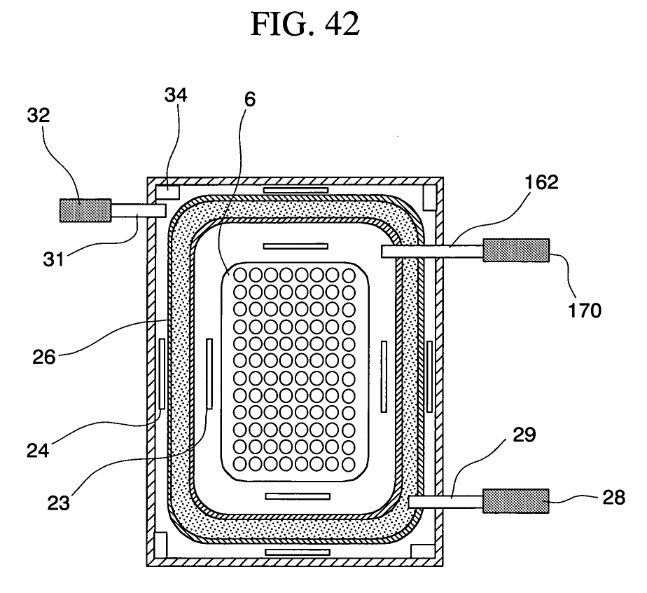

FIG. 42 shows an example in which a well plate is used for the culturing vessel 184.

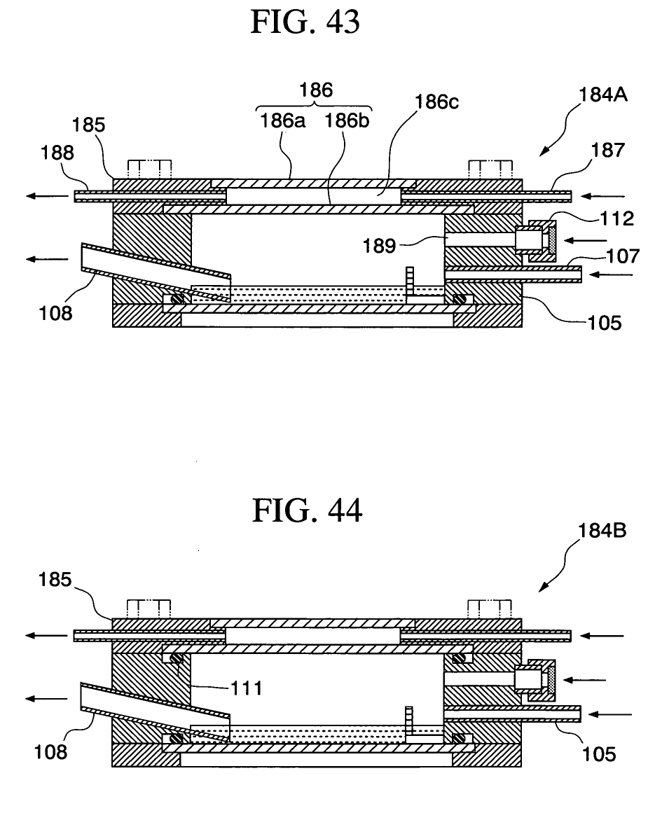

In addition to the culturing vessels used in the first and second embodiments, an atmospherically open culturing vessel 184A may be used for the atmospherically open culturing vessel 184 that is used in the third embodiment. As shown in FIG. 43, a top glass 186 that is mounted on a top glass fixing member 185 of the atmospherically open culturing vessel 184A has what is known as a pair glass structure that is formed by a pair of glass plates 186a and 186b that are mounted respectively on the top surface and the bottom surface of the top glass fixing member 185. A sealed space 186c is formed between the pair of glass plates 186a and 186b. A warm air supply port 187 and a warm air discharge port 188 are provided in the top glass fixing member 185 so as to communicate with the sealed space 186c. The warm air supply tube 176 is connected to the warm air supply port 187.

The culture solution supply port 107 and a warm moisture intake aperture 189 are both provided in the central member 105. When carbon dioxide is introduced into the culturing vessel, it is introduced via the warm moisture intake aperture 189 when it is introduced in the form of a gas directly into the incubator 161. When it is mixed into culture solution, it is introduced together with the culture solution via the culture solution supply port 107.

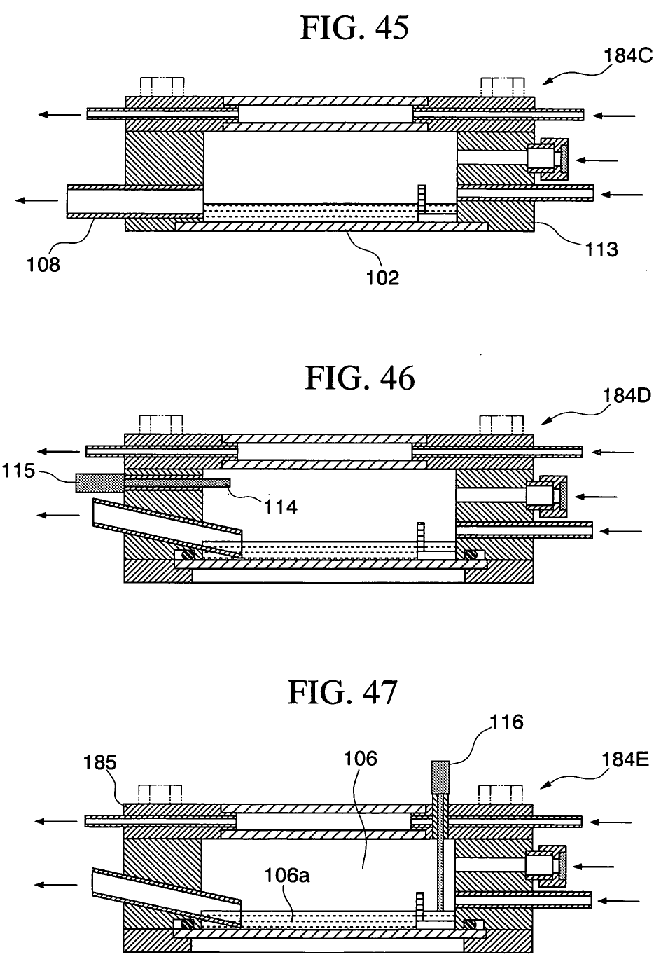

Other examples of atmospherically open culturing vessels will now be described. As shown in FIG. 44, in a culturing vessel 184B, the top glass fixing member 185 is mounted via an O ring 111 on the central member so as to hermetically seal the vessel. As shown in FIG. 45, in a culturing vessel 184C there is provided a common central member that obtained by uniting the bottom glass fixing member and the central member. The bottom glass 102 is adhered to a bottom portion of the common central member 113 so as to hermetically seal the vessel.

As shown in FIG. 46, a carbon dioxide supply coupling 114 that guides carbon dioxide inside the culturing vessel to the outside of the culturing vessel, and a carbon dioxide sensor 115 that is mounted on an exterior end of the carbon dioxide supply coupling 114 are in a culturing vessel 184D. Using the carbon dioxide sensor 115, it is possible to detect the concentration of carbon dioxide inside the culturing vessel.

As shown in FIG. 47, a liquid level sensor 116 is mounted on the top glass fixing member 185 of a culturing vessel 184E. As a result, it is possible to detect the height of the surface of the culturing solution phase 106a inside the culturing vessel 106.

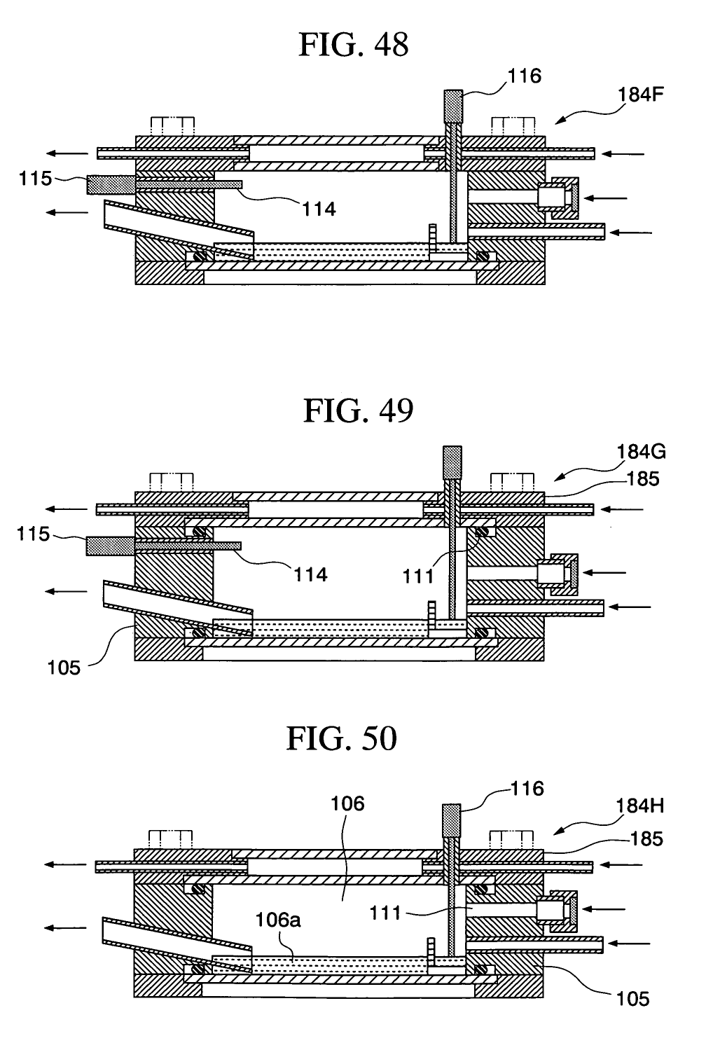

As shown in FIG. 48, the carbon dioxide supply coupling 114, the carbon dioxide sensor 115, and the liquid level sensor 116 are mounted on a culturing vessel 184F.

As shown in FIG. 49, the top glass fixing member 185 is mounted on the central member 105 of a culturing vessel 184G via an O ring 111 such that the vessel is hermetically sealed. Furthermore, the carbon dioxide supply coupling 114 and the carbon dioxide sensor 115 are provided in the culturing vessel 184G so that the concentration of carbon dioxide inside the culturing vessel can be detected.

As shown in FIG. 50, the top glass fixing member 185 is mounted on the central member 105 of a culturing vessel 184H via an O ring 111 such that the vessel is hermetically sealed. Furthermore, the liquid level sensor 116 is provided in the culturing vessel 184H so that the height of the surface of the culture solution layer 106a inside the culturing chamber 106 can be detected.

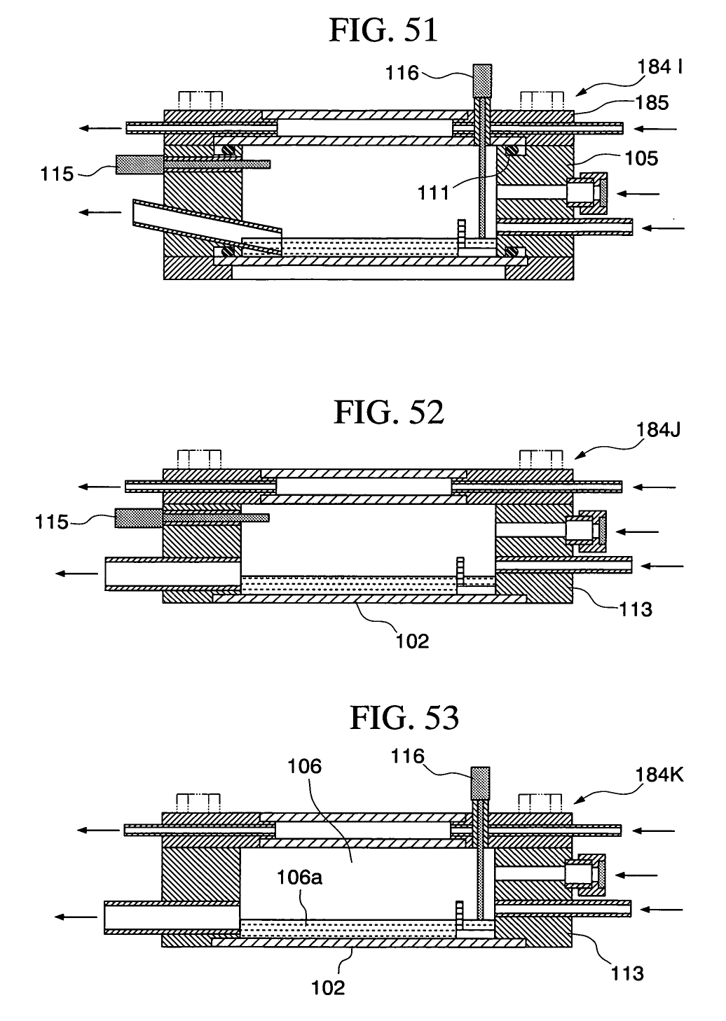

As shown in FIG. 51, the top glass fixing member 185 is mounted on the central member 105 of a culturing vessel 184I via an O ring 111 such that the vessel is hermetically sealed. Furthermore, the carbon dioxide sensor 115 and the liquid level sensor 116 are provided in the culturing vessel 184I.

As shown in FIG. 52, a common central member 113 that is formed by integrating the bottom glass fixing member with the central member is provided in a culturing vessel 184J. The bottom glass 102 is adhered to a bottom portion of the common central member 113 so as to hermetically seal the vessel. Furthermore, the carbon dioxide sensor 115 is provided in the culturing vessel 184J so that the concentration of carbon dioxide inside the culturing vessel can be detected.

As shown in FIG. 53, a common central member 113 that is formed by integrating the bottom glass fixing member with the central member is provided in a culturing vessel 184K. The bottom glass 102 is adhered to a bottom portion of the common central member 113 so as to hermetically seal the vessel. Furthermore, the liquid level sensor 116 is provided in the culturing vessel 184K so that the height of the surface of the culture solution layer 106a inside the culturing chamber 106 can be detected.

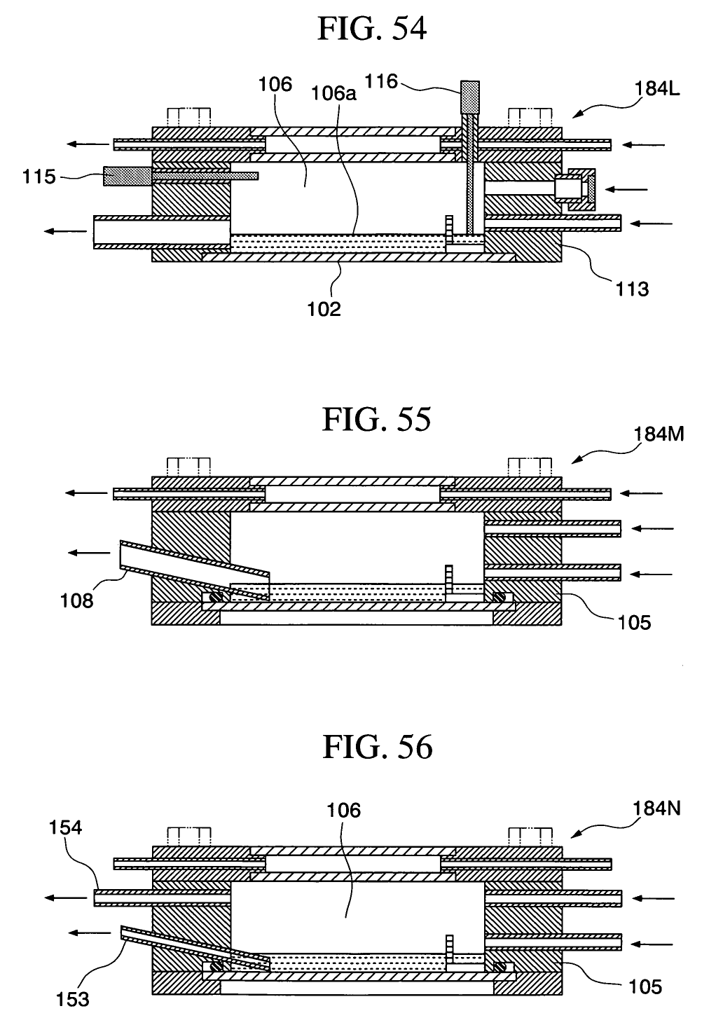

As shown in FIG. 54, a common central member 113 that is formed by integrating the bottom glass fixing member with the central member is provided in a culturing vessel 184L. The bottom glass 102 is adhered to a bottom portion of the common central member 113 so as to hermetically seal the vessel. Moreover, the carbon dioxide sensor 115 is provided in the culturing vessel 184L so that the concentration of carbon dioxide inside the culturing vessel can be detected. Furthermore, the liquid level sensor 116 is provided in the culturing vessel 184L so that the height of the surface of the culture solution layer 106a inside the culturing chamber 106 can be detected.

As shown in FIG. 55, the cap 112 that is provided with an anti-bacterial filter is not provided on the warm moisture intake aperture of a culturing vessel 184M. Instead, an anti-bacterial filter is provided on the carbon dioxide supply tube 170 and the warm air supply tube 176 and the like that are on the flow path side.

As shown in FIG. 56, the waste solution discharge port 153 and the air discharge port 154 are provided separately in the central member 105 of a culturing vessel 184N. Namely, old culture solution is discharged to the outside of the culturing chamber 106 via the waste solution discharge tube 87 that is connected to the waste solution discharge port 153, while the air inside the culturing chamber 106 is discharged to the outside through the air discharge port 154.

The diameter of the waste solution discharge port 153 is smaller than that of the waste solution discharge port 108 that is used in the culturing vessels shown in FIGS. 43 through 55 and simultaneously discharges both air and waste solution. Moreover, the air discharge port 154 is located in the top portion of the culturing chamber 106.

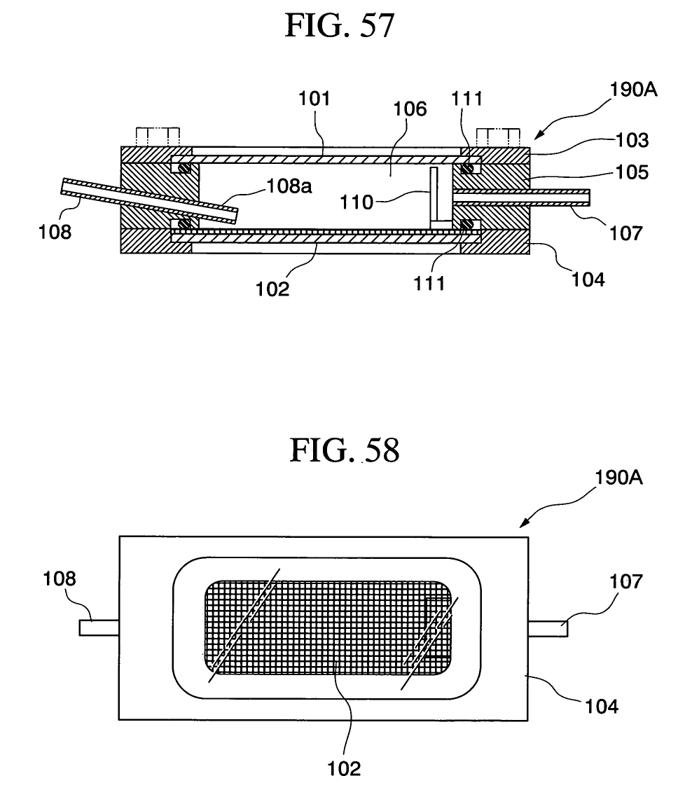

Next, a description will be given of a sealed type of culturing vessel 190 that is used in the third embodiment. As shown in FIGS. 57 and 58, a sealed type of culturing vessel 190A is provided with a top glass 101 and a bottom glass 102 that have optical characteristics and are positioned apart from each other in a vertical direction, a top glass fixing member 103 and a bottom glass fixing member 104 that respectively fix the top glass 101 and the bottom glass 102, and a central member 105 that is placed between the top glass fixing member 103 and the bottom glass fixing member 104 and forms side walls of the culturing vessel. A culturing chamber 106 is formed inside the culturing vessel 190A. In FIG. 58, the outer configuration of the culturing vessel 190A is a square configuration. However, it is not essential that the outer configuration thereof be a square configuration and, for example, the outer configuration may be a round configuration or a hexagonal configuration or the like.

The central member 105 is provided with a culture solution supply port 107 that is connected to the culture solution supply tube 56 and guides culture solution that is supplied from the culture solution supply tube 56 to the interior of the culturing vessel, a waste solution discharge port 108 that is connected to the waste solution discharge tube 87 and discharges old culture solution from the waste solution discharge tube 87, and a commutation member 110 that adjusts the flow of culture solution.

Glass that has been AR coated in portions corresponding to the surface on the culturing chamber 106 side or glass that has received a hydrophilic coating in portions corresponding to the surface on the culturing chamber 106 side may be used for the top glass 101.

The bottom glass 102 may also be provided with a reinforcing structure in order to protect the glass from breakages that are caused by physical factors such as, for example, pressure and temperature changes when a fluid such as culture solution and the gas that is required for culturing are supplied to the interior of the culturing vessel.

The top glass 101 is fixed to the top glass fixing member 103 by being inserted therein or by adhesion, while the bottom glass 102 is fixed to the bottom glass fixing member 104 by being inserted therein or by adhesion. The top glass fixing member 103 and the bottom glass fixing member 104 are fixed to the central member 105 such that the vessel is hermetically sealed via an O ring 111.

In the mutual relationship between the top glass fixing member 103 and bottom glass fixing member 104 and the central member 105, by employing, for example, an assembly structure that uses screws or a fit-together structure, it becomes possible to disassemble the top glass fixing member 193, the bottom glass fixing member 104, and the central member 105. Moreover, the respective members forming the culturing vessel 190A, namely, the top glass fixing member 103, the bottom glass fixing member 104, the central member 105, the culture solution supply port 107, and the commutation member 110 are made from a material that is not toxic to cells such as, for example, PEEK, PPS, or PSF.

The culture solution supply port 107 is placed in a position lower than the commutation member 110.

A culture solution suction portion 108a of the waste solution discharge port 108 is positioned slightly above the observation surface. The culture solution supply port 107 and the waste solution discharge port 108 can be blocked off by plugs such as rubber caps. Note that the structure of these plugs may be such that they are assembled in advance together with the culture solution supply port 107 and the waste solution discharge port 108, and the blocked off state of these apertures can be ended automatically when the culture solution supply tube 56 and the waste solution discharge tube 87 are connected respectively thereto.

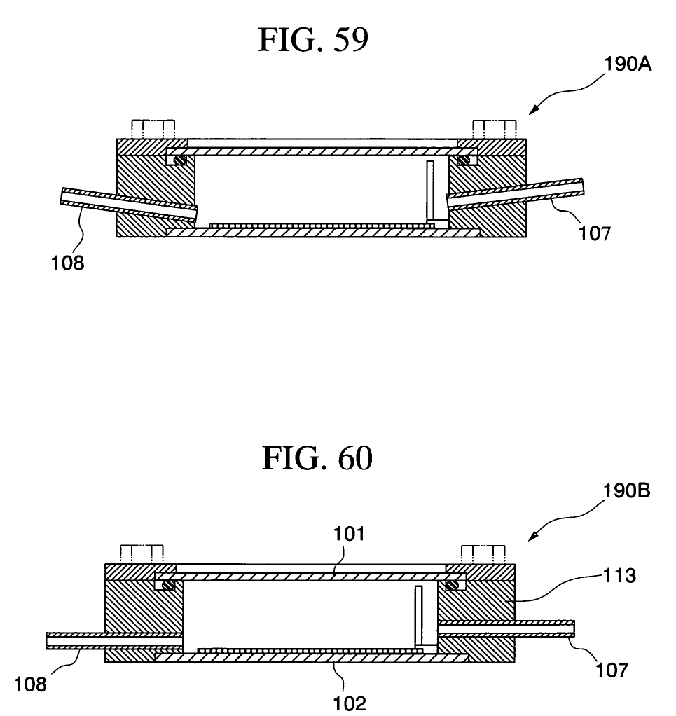

Note that, in FIG. 57, only the waste solution discharge port 108 is mounted on a slant. However, as shown in FIG. 59, it is also possible for the culture solution supply port 107 and the waste solution discharge port 108 to both be mounted on a slant such that end portions thereof that are located on the exterior side of the culturing vessel 190A are positioned higher than end portions thereof that are positioned on the interior side of the culturing vessel 190A. By employing this type of structure, it is possible to prevent the solution inside the culturing vessel from being spilled when the vessel is being transported.

Note that the culture solution supply port 107 and the waste solution discharge port 108 can also be used as cell suspension solution supply ports to supply cell suspension solution using a pipette or the like. The commutation member 110 has a lattice configuration in which are formed a plurality of gaps that are made by constructing grooves or columnar protrusions having a depth or height of, for example, 0.5 to 1 mm.

Other examples of a sealed type of culturing vessel will now be described. The culturing vessel 190B shown in FIG. 60 is provided with a common central member 113 that is formed by integrating the bottom glass fixing member with the central member. The bottom glass 102 is, for example, adhered to a bottom portion of the common central member 113 so as to hermetically seal the vessel. Moreover, the culture solution supply port 107 and the waste solution discharge port 108 are not tilted and are substantially parallel to the top glass 101 and the bottom glass 102.

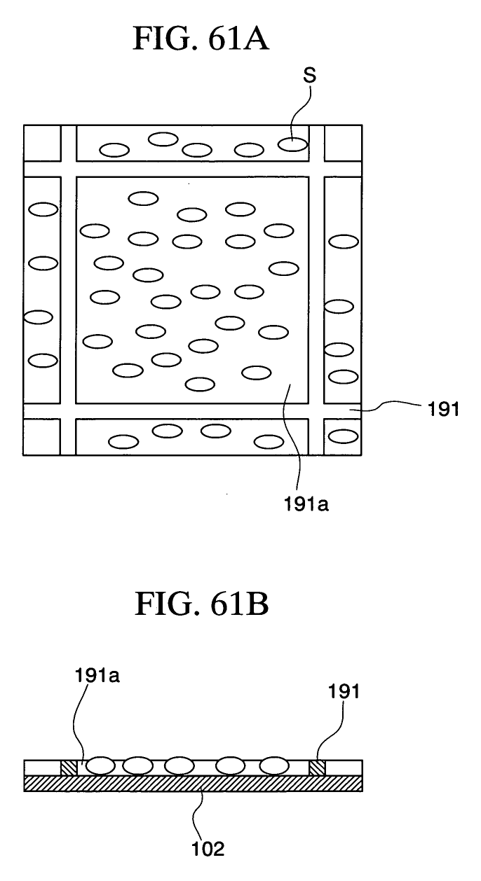

A lattice-shaped structural member (for example, a lattice member 191) may be fixed to the bottom glass 102 in order to reinforce the bottom glass 102. In this case, as shown in FIGS. 61A and 61B, the thickness of the lattice member 191 may be set at a thickness that corresponds to the height that is lower than the cells S and also lower than the surface of the culture solution on the inner side (the top surface) of the bottom glass 102.

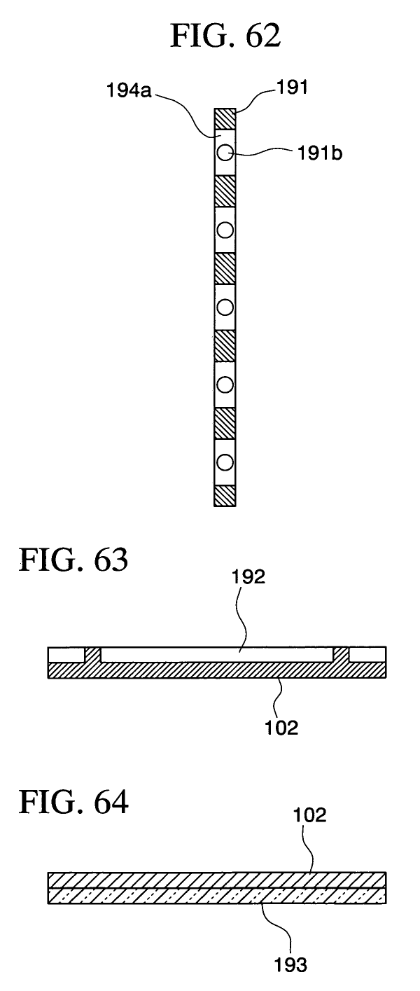

The lattice member 191 may be fixed in a state of submergence in the culture solution. In this case, as shown in FIG. 62, one or more holes 191b are provided as flow paths in each lattice member 191a. These holes 191b make it possible to prevent culture solution from accumulating in the vicinity of the lattice member 191.

By employing the above described structure, culture solution can easily spread to the cells S that adhere to the glass surface inside the lattice. Accordingly, because any bias in the supply of culture solution to the cells S can be controlled and it is easier to remove waste material such as lactic acid from the cells S, it is possible to prevent the cells S from being affected.

Moreover, it is not essential for the lattice member 191 to be provided on the inner surface side of the bottom glass 102, and it may also be provided on the outer surface side of the bottom glass 102. In this case, because the lattice member 191 is not provided on the same surface as the surface where the cells S are located, bias occurs in the supply of culture solution to the cells S. Accordingly, it is possible to prevent the cells S from being affected.

If the lattice member 191 is provided on the inner surface side of the bottom glass 120, the objective lens that is used for observing the cells S can be placed as close as possible to the bottom glass 102. This makes it possible for an objective lens having a large numerical aperture to be used to observe the cells S and makes it possible for even more reliable observation results to be obtained.

If the lattice member 191 is not provided on the outer surface side of the bottom glass 102, then it is possible to control the occurrence of any optical interference from the lattice member 191 towards the image of the cells S when the cells S are observed using an objective lens.

Note that a slide glass or cover glass may also be used for the bottom glass 102 that is used in the present embodiment instead of the glass that is normally used in a culturing vessel.

Moreover, instead of fixing the lattice member 191 to the bottom glass 102, as shown in FIG. 63, it is also possible to form lattice-shaped grooves 192 directly in the bottom glass 102. If the lattice-shaped grooves 192 are formed directly in the top surface of the bottom glass 102, then it is desirable that the depth of the grooves is lower than the height of the cells and is also lower than the surface of the culture solution.

By employing this type of structure, in the same way as the embodiment shown in FIGS. 61A and 61B, bias occurs in the supply of culture solution to the cells S. Accordingly, it is possible to prevent the cells S from being affected.

The structure to reinforce the bottom glass 102 is not limited to having a lattice-shaped configuration, as is the case with the lattice member 191, and, as shown in FIG. 64, it is also possible to adhere a reinforcing sheet 193 that has optical characteristics to the bottom surface of the bottom glass 102. By employing this type of structure, the possibility of bias occurring in the supply of culture solution to the cells S is reduced.

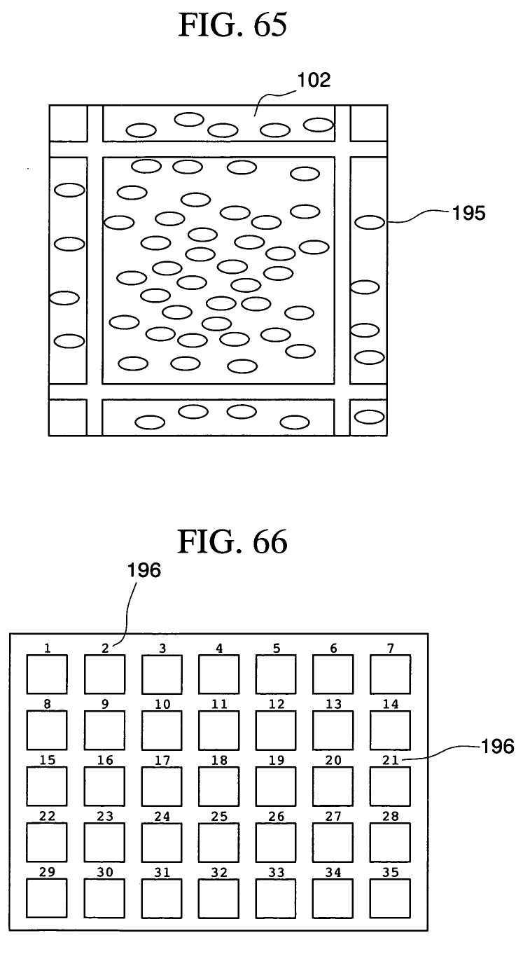

If the bottom glass 102 is reinforced using a lattice structure such as that shown in FIGS. 61A through 64, then it is preferable that the framework for the lattices is set to a size that allows it to be transferred onto a photograph area (within the field of vision) 195, as shown in FIG. 65. This enables any obstruction of the field of vision of the microscope during an observation of the cells S to be prevented.

Furthermore, if a lattice-shaped reinforcing member is used to reinforce the bottom glass 102, as shown in FIG. 66, it is preferable that symbols 196 such as numbers or characters or the like for position identification are stamped or printed on each lattice. As a result, such symbols can be verified inside the field of vision when an observation is being made using a microscope or the like, so that the position of the cell being observed can be easily specified.

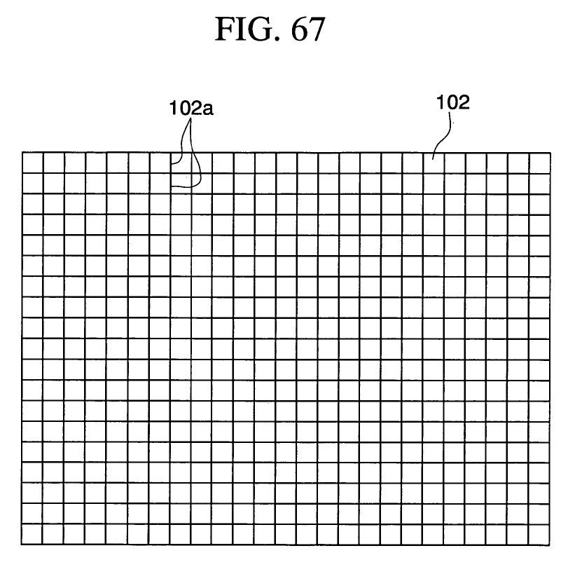

Other examples of methods of reinforcing the bottom glass 102 include, for example, embedding lattice-shaped wire 102a inside the bottom glass 102 as shown in FIG. 67. By employing this method, bumps and indentations are not created in the surface of the bottom glass 102 so that, consequently, optical interference with an image of the cells S as well as the possibility of bias occurring in the supply of culture solution to the cells S can be reduced.

A description of the operation of the biological sample culturing and observation system having the above described structure will now be given with reference made to FIG. 41. Sterilized water that is stored in the humidification water tank 49 is suctioned by the humidification water supply pump 86 and is supplied via the humidification water supply tube 28 to the interior of the incubator 161. Accordingly, the interior of the incubator 161 is kept at a suitable humidity.

When supplying carbon dioxide to the culturing vessel, a method may be used in which the carbon dioxide is supplied indirectly to the culturing vessel 184 by being supplied to the interior of the incubator 161, or a method in which the carbon dioxide is supplied directly to the interior of the culturing vessel 184.

Namely, after carbon dioxide that is stored in the carbon dioxide tank 82 has been suctioned by the air pump 166 and has been warmed to a suitable temperature by the heater 168, it is sent to the flow path switching valve 169. If an atmospherically open type of culturing vessel is being used, the carbon dioxide that has been warmed to a suitable temperature is supplied to the interior of the incubator 161 by the flow path switching valve 169. As a result, carbon dioxide is supplied indirectly to the interior of the atmospherically open culturing vessel 184.

By providing the liquid level sensor 116 in the culturing vessels 184E to 184I, 184K, and 184L that are used, it becomes possible to control the volume of solution. As a result, it becomes possible to provide advance warning of faults such as leaks and the like.

Furthermore, by providing the carbon dioxide sensor 115 in the culturing vessels 184D, 184F, 184G, 184I, 184J, and 184L, the concentration of carbon dioxide inside the culturing chamber 106 can be monitored and feedback therefrom can be provided to the carbon dioxide supply side. As a result, the optimum concentration of carbon dioxide can be set constantly in the culturing chamber 106.

If, however, a sealed type of culturing vessel is used, then by further supplying carbon dioxide that has been warmed to an appropriate temperature by the heater 168 to the flow path switching valve 172, and here switching the flow path thereof, this carbon dioxide can be supplied to the interior of the appropriately selected reagent vessel 44, 45, 46, and 48. In this manner, the carbon dioxide is mixed, for example, with the culturing solution inside the reagent vessels 44, 45, 46, and 48 and is supplied with the respective culturing solutions directly to the interior of the culturing vessel.

At this time, during a discharge of old solution from the interior of the culturing vessel, if the suctioned culture solution is supplied to the reagent vessel 44, 45, 46, or 48 that is supplying the culturing vessel by switching the flow path switching valve 180, then a circulatory form of culture solution supply becomes possible.

Because the flow path switching valves 169 and 172 that switch the carbon dioxide flow path and the switching valve 180 that switches the waste solution flow path are provided, it is possible to selectively supply carbon dioxide to the reagent vessels 44, 45, 46, and 48 and circulate the culture solution by switching the flow path of the culture solution to the reagent vessels. As a result, in addition to the atmospherically open culturing vessels described in the first and second embodiments, culturing is possible using sealed culturing vessels in which the vessel interior is filled with culture solution. Accordingly, a variety of experiments and observations can be made using a variety of vessels.

Note that because a gradual change in environment is caused by the circulation of culture solution in a sealed culturing vessel, it can be used for protocols where external factors are to be excluded as much as possible, and also for protocols where contamination is to be completely excluded.

Moreover, because the bottom glass 102 of the sealed culturing vessel that is used in the third embodiment is reinforced by a lattice member or wire or the like, the effect is obtained that it is possible to control any damage or flexure that is caused by changes in pressure when culture solution is supplied.

Furthermore, a structure is employed that makes it possible to select whether the clean air that is stored in the clean air tank 63 is to be supplied, using the air switching valve 175, to the common culture solution supply tube 58 side or to the warm air supply tube 176 side. If the atmospherically open culturing vessel shown in FIGS. 38 through 51 is used, then clean air can be supplied to the warm air supply tube 176 side by switching the air switching valve 175 and can be supplied to the sealed space inside the pair glass of the atmospherically open culturing vessel. As a result, the top and bottom glass are warmed and it is possible to prevent condensation from forming on the top glass and prevent heat from being lost via the top glass. As a result, stable culturing in which any change in the temperature in the culturing chamber is suppressed can be performed. Furthermore, in a phase contrast observation or the like that uses transmitted illumination, an image that has excellent contrast can be acquired.

If the sealed culturing vessel shown in FIGS. 57 to 60 is used, then clean air can be supplied to the common culture solution supply tube 58 side by switching the air switching valve 175, and the clean air can thus be supplied to the interior of the sealed culturing vessel.

As a result, because the interior of the common culture solution supply tube 58 is filled with air when the solution is being switched, it is possible to prevent a greater quantity of culture solution than is required being supplied to the interior of the culturing vessel. Moreover, by supplying air when culture solution is being supplied or replaced, the flow rate of culture solution to the culturing vessel can be adjusted. Therefore, gentle culturing can be achieved without any pressure being applied to the cells.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as limited by the foregoing description and is only limited by the scope of the appended claims.

What is claimed is:

1. A biological sample culturing and observation system in which temporal changes in a cultured biological sample are observed while the biological sample is being cultured, comprising:
an incubator in which the biological samples are housed, and whose interior is maintained in a culturing environment satisfying predetermined conditions, and the interior is isolated from the outside;
an observation optical system that from outside the incubator optically observes the biological sample via the incubator;
a light blocking device that blocks external light that is irradiated on the biological sample and within the visual field of the observation optical system; and
a supply device that selectively supplies a liquid or gas to the biological sample inside the incubator from a plurality of holding vessels that individually hold a plurality of different types of liquid or gas that are necessary for the culturing.

2. The biological sample culturing and observation system according to claim 1, wherein
the incubator comprise a light transmitting portion that prevents any conduction between the environment inside the incubator and the environment outside the incubator, and that transmits light that is necessary for the optical observation between the observation optical system and the biological sample.

3. The biological sample culturing and observation system according to claim 1, wherein at least one of the holding vessels holds culture gas which is necessary for the culturing.

4. The biological sample culturing and observation system according to claim 3, further comprising a culture gas sensor that measures a concentration of the culture gas that is supplied to the interior of the incubator.

5. The biological sample culturing and observation system according to claim 3, wherein
the supply device comprises a flow path switching valve that switches a flow path from the holding vessel from which the culture gas is supplied between a state in which the flow path is connected to the interior of the incubator and a state in which the flow path is connected to the interior of another of the holding vessels.

6. The biological sample culturing and observation system according to claim 1, further comprising a water tank that holds a liquid that is used to humidify the interior of the incubator.

7. The biological sample culturing and observation system according to claim 6, wherein
the supply device comprises a culture gas mixing device that mixes culture gas which is necessary for the culturing, in the humidifying liquid that is supplied to the water tank.

8. The biological sample culturing and observation system according to claim 1, wherein
the supply device comprises a heater that warms the liquid or the gas that is supplied to the biological sample to a temperature that is required for the culturing.

9. The biological sample culturing and observation system according to claim 1, wherein
the supply device connects a waste solution discharge flow path that discharges a liquid that is supplied to the biological sample to at least one of a space inside a holding vessel that holds a liquid to be supplied to the biological sample or a space inside a waste solution tank that holds the discharged liquid.

10. The biological sample culturing and observation system according to claim 9, wherein
the supply device is provided with a flow path switching valve that switches the waste solution discharge flow path between a state in which the flow path is connected to a space inside a holding vessel that holds a liquid to be supplied to the biological sample and a state in which the flow path is connected to a space inside the waste solution tank.

11. The biological sample culturing and observation system according to claim 1, further comprising a control unit that sets replacement periods for replacing the liquid supplied to the biological sample with new liquid relative to the supply device.

12. The biological sample culturing and observation system according to claim 11, wherein
the control unit sets replacement periods for replacing the liquid supplied to the biological sample with new liquid based on the replacement period that is selected from a table that is input in advance into the control unit.

13. The biological sample culturing and observation system according to claim 11, wherein
the control unit sets replacement periods for replacing the liquid supplied to the biological sample with new liquid based on natural fluorescence or a degree of light absorption of the liquid that is detected.

14. The biological sample culturing and observation system according to claim 1, wherein
the incubator houses a culturing vessel comprising a culturing section that holds the biological sample being cultured, and a light transmitting section that transmits light that is required for the optical observation onto an optical path between the observation optical system and the biological sample that is held in the culturing section.

15. The biological sample culturing and observation system according to claim 14, wherein
the supply device comprises a flow path switching valve that switches the flow path such that at least one of the plurality of holding vessels is connected to the culturing vessel, and that valve is disposed on a flow path that connects a space inside each of the holding vessels to a space inside the culturing vessel.

16. The biological sample culturing and observation system according to claim 15, wherein
at least one of the holding vessels holds humidifying water that is used to humidify the biological sample, and
the flow path switching valve switches between a state in which moisture which is required for the culturing and is generated by the humidifying water is supplied to the space inside the incubator and a state in which the moisture is supplied to a space inside the culturing vessel.

17. The biological sample culturing and observation system according to claim 15, wherein
at least one of the holding vessels holds a culture gas which is necessary for the culturing, and the flow path switching valve switches between a state in which a flow path from the holding vessel holding the culture gas is connected to the space inside the incubator and a state in which the flow path is connected to the space inside the culturing vessel.

18. The biological sample culturing and observation system according to claim 15, wherein
the flow path switching valve switches the flow path such that the space inside the holding vessel is connected to the space inside the culturing vessel based on detection results of at least one of the state of the degree of light absorption or the state of the natural fluorescence in the culture solution that is supplied to the biological sample.

19. The biological sample culturing and observation system according to claim 15, wherein
the flow path switching valve further comprises a spare port that is used to add and connect another of the holding vessels.

20. The biological sample culturing and observation system according to claim 14, wherein
the supply device comprises a supply pumps that supply the liquid or the gas inside the holding vessels to the culturing vessel, and that are located on the flow path that connects the spaces inside plurality of holding vessels respectively to the space inside the culturing vessel, and wherein
the supply pumps are independently located on each of the holding vessels.

21. The biological sample culturing and observation system according to claim 14, wherein
the supply device comprises an blocking air injection device that injects air in order to block the liquid being supplied from the holding vessel to the culturing vessel, and that is located on the flow path that connects the space inside the holding vessel that holds the liquid required for the culturing to the space inside the culturing vessel.

22. The biological sample culturing and observation system according to claim 14, wherein
the light transmitting portion on at least one surface of the culturing vessel includes a structure in which there are provided a pair of transparent plates that allow the light that is required for the optical observation to be transmitted.

23. The biological sample culturing and observation system according to claim 14, wherein
a strengthening device that improves the strength of the transparent plates is provided on the light transmitting portion on at least one surface of the culturing vessel.

24. The biological sample culturing and observation system according to claim 14, wherein
the culturing vessel further comprises a commutation member that commutates the flow of a liquid that is supplied to the interior of the culturing vessel.

25. The biological sample culturing and observation system according to claim 14, further comprising a transporting case that holds the plurality of holding vessels whose internal spaces connected through a flow path to the space inside the culturing vessel, and that simultaneously transports each of the holding vessels.

26. The biological sample culturing and observation system according to claim 14, wherein
the culturing vessel is composed of a biological sample containing vessel that includes the culturing section and the light transmitting section, and a lid that covers the culturing section in the biological sample containing vessel, and wherein
the lid comprises a supply aperture that guides the gas that is required for the culturing and is supplied from the supply device and a discharge aperture that discharges the gas that is supplied inside the culturing section to the outside of the culturing section.

27. The biological sample culturing and observation system according to claim 1, wherein
the observation optical system comprises a stage that moves in at least two intersection axial directions, and wherein the incubator is mounted on the stage.

28. The biological sample culturing and observation system according to claim 1, wherein the supply device further comprises a chilling device that refrigerates or freezes the liquid that is held inside the holding vessel, and a warming device that warms a liquid when the liquid is supplied to the biological sample to a temperature that is required for the culturing.

29. The biological sample culturing and observation system according to claim 1, further comprising a thermal insulating device to keep an objective lens in the observation optical system at the temperature that is required for the culturing.

30. The biological sample culturing and observation system according to claim 28, further comprising a heat conductivity control device that controls the conduction of heat between an environment in which the objective lens is insulated and an environment in which the biological sample is cultured.

31. The biological sample culturing and observation system according to claim 1, further comprising a liquid level detecting device that detects a level of liquids held inside the holding vessels.

32. An incubator that is used in a biological sample culturing and observation system in which temporal changes in a cultured biological sample are optically observed using an observation optical system while the biological sample is being cultured, wherein
the biological sample culturing and observation system comprising:
an observation optical system that optically observes the biological sample;
a light blocking device that blocks external light that is irradiated on the biological sample and within the visual field of the observation optical system; and
a supply device that selectively supplies a liquid or gas to the biological sample from a plurality of holding vessels that individually hold a plurality of different types of liquid or gas that are necessary for culturing the biological sample, and wherein
the biological sample that is optically observed from the outside using the observation optical system is contained inside the incubator, and the biological sample is cultured by maintaining the interior of the incubator isolated from the outside in an environment fulfilling predetermined conditions.

33. A supply device for liquid or gas that is used in a biological sample culturing and observation system in which temporal changes in a cultured biological sample are observed while the biological sample is being cultured, wherein
the biological sample culturing and observation system comprising:
an incubator whose interior is maintained in a culturing environment whose conditions have been predetermined and that is isolated from the outside, with the biological samples being housed in this interior;
an observation optical system that from outside the incubator optically observes the biological sample via the incubator; and
a light blocking device that blocks external light that is irradiated on the biological sample and is within the visual field of the observation optical system, and wherein
the liquid or gas is selectively supplied to the biological sample contained inside the incubator from a plurality of holding vessels that individually hold a plurality of different types of liquid or gas that are necessary for the culturing.

34. A culturing vessel that is contained inside the incubator of a biological sample culturing and observation system in which temporal changes in a cultured biological sample are observed while the biological sample is being cultured, wherein
the biological sample culturing and observation system comprising:
an incubator whose interior is maintained in a culturing environment whose conditions have been predetermined and that is isolated from the outside, with the biological samples being housed in this interior;
an observation optical system that from outside the incubator optically observes the biological sample via the incubator;
a light blocking device that blocks external light that is irradiated on the biological sample and within the visual field of the observation optical system; and
a supply device that selectively supplies a liquid or gas to the biological sample contained inside the incubator from a plurality of holding vessels that individually hold a plurality of different types of liquid or gas that are necessary for culturing the biological sample, and wherein
the culturing vessel comprises:
a culturing section that holds the biological sample;
a light transmitting section that transmits light that is necessary for the optical observation onto an optical path between the observation optical system and the biological sample that is held in the culturing section;
a first flow path that supplies the liquid or the gas from the supply device to the biological sample that is held in the culturing section; and
a second flow path that discharges the liquid or gas that was supplied to the biological sample to the outside.
